SEQUENCE LISTING (2 sequences)

SEQ ID NO: 1 - DNA, 1254 bp, Homo sapiens
atgctgctatccgtgccgctgctgctcggcctcctcggcctggccgtcgccgagcctgccgtctacttcaaggagcagtttctggacggagacgggtggacttcccgctggatcgaatccaaacacaagtcagattttggcaaattcgttctcagttccggcaagttctacggtgacgaggagaaagataaaggtttgcagacaagccaggatgcacgcttttatgctctgtcggccagtttcgagcctttcagcaacaaaggccagacgctggtggtgcagttcacggtgaaacatgagcagaacatcgactgtgggggcggctatgtgaagctgtttcctaatagtttggaccagacagacatgcacggagactcagaatacaacatcatgtttggtcccgacatctgtggccctggcaccaagaaggttcatgtcatcttcaactacaagggcaagaacgtgctgatcaacaaggacatccgttgcaaggatgatgagtttacacacctgtacacactgattgtgcggccagacaacacctatgaggtgaagattgacaacagccaggtggagtccggctccttggaagacgattgggacttcctgccacccaagaagataaaggatcctgatgcttcaaaaccggaagactgggatgagcgggccaagatcgatgatcccacagactccaagcctgaggactgggacaagcccgagcatatccctgaccctgatgctaagaagcccgaggactgggatgaagagatggacggagagtgggaacccccagtgattcagaaccctgagtacaagggtgagtggaagccccggcagatcgacaacccagattacaagggcacttggatccacccagaaattgacaaccccgagtattctcccgatcccagtatctatgcctatgataactttggcgtgctgggcctggacctctggcaggtcaagtctggcaccatctttgacaacttcctcatcaccaacgatgaggcatacgctgaggagtttggcaacgagacgtggggcgtaacaaaggcagcagagaaacaaatgaaggacaaacaggacgaggagcagaggcttaaggaggaggaagaagacaagaaacgcaaagaggaggaggaggcagaggacaaggaggatgatgaggacaaagatgaggatgaggaggatgaggaggacaaggaggaagatgaggaggaagatgtccccggccaggccaaggacgagctgtaa

SEQ ID NO: 2 - protein, 417 aa, Homo sapiens
MLLSVPLLLGLLGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAKDEL

(19) United States
(12) Patent Application Publication
(10) Pub. No.: US 2021/0169979 A1
(43) Pub. Date: Jun. 10, 2021

(54) SYSTEM AND METHOD FOR SONOSENSITIZED CANCER IMMUNOTHERAPY WITH NANOPARTICLES

(71) Applicant: The Board of Regents for the Oklahoma Agricultural and Mechanical Colleges, Stillwater, OK (US)

(72) Inventors: Ashish Ranjan, Stillwater, OK (US); Sri Nandhini Sethuraman, Stillwater, OK (US); Mohit Pratap Singh, Stillwater, OK (US); Jerry R. Malayer, Stillwater, OK (US)

(21) Appl. No.: 17/113,833

(22) Filed: Dec. 7, 2020

Related U.S. Application Data
(60) Provisional application No. 62/944,098, filed on Dec. 5, 2019.

(51) Int. Cl. A61K 38/17; A61K 9/127; A61K 41/00; A61K 45/06; A61N 7/02

(52) U.S. Cl. CPC A61K 38/1738; A61K 9/127; A61N 7/02; A61K 45/06; A61K 41/0028

(57) ABSTRACT

Compositions, kits, and systems are disclosed that include calreticulin gene delivering nanoparticles that can be used alone or in combination with focused ultrasound to induce immunogenic cell death in tumors. Also disclosed are methods of producing and using the compositions, kits, and systems.

### CROSS REFERENCE TO RELATED APPLICATIONS/INCORPORATION BY REFERENCE STATEMENT

This application claims benefit under 35 USC § 119(e) and 35 USC § 21 of U.S. Ser. No. 62/944,098, filed Dec. 5, 2019. The entire contents of the above-referenced patent(s)/patent application(s) are hereby expressly incorporated herein by reference.

### STATEMENT REGARDING FEDERALLY SPONSORED RESEARCH OR DEVELOPMENT

Not Applicable.

### BACKGROUND

The ability of the immune system to recognize, attack, and eliminate tumors is well established. However, several tumor types (such as, but not limited to, melanoma, pancreatic, and breast tumor types) manifest a variety of immunosuppressive mechanisms to evade immune attack. As such, reversing the protective immune suppression mechanisms and stimulating immune cell activation is the fundamental goal of cancer immunotherapy. Conventional chemo- and radiotherapy can contribute to immunostimulation when they kill cells in a specific manner that releases damage associated molecular patterns (DAMPS), such as (but not limited to) calreticulin (CRT), ERP57, HMGB1, ATP, and heat shock proteins, from dying cells. These mediators, which are normally only found internally in cells, are recognized by receptors on immune cells and are strongly immunostimulatory when found extracellularly or on the outside surface of the dying cell membrane. This phenomenon is called immunogenic cell death (ICD), and when tumor cells experience ICD, the immunostimulatory effects of ICD can prime and improve the populations of tumor antigen presenting and cytotoxic T-cells (CTLs), thereby enhancing antitumor immune responses.

For instance, an appropriate administration of thermal or chemotherapy stress to cancer cells causes ICD in which CRT translocates to the cell surface to enhance phagocytosis and immunogenic recognition of dying cancer cells by antigen presenting cells (APCs). CRT also impacts blood vessels by augmenting the expression of adhesion molecules such as ICAM-1 and VCAM-1 on tumor endothelial cells, thus enhancing the efficiency of their interactions with tumor-infiltrating leukocytes. Like tumor cells, tumor-associated macrophages expressing activated CRT on cell surfaces exhibit efficient recognition of cancerous cells for phagocytosis.

Although able to contribute to the needed immune stimulation, CRT expression is often inconsistent in most tumors and generally is not sufficient to generate an effective antitumor immune response on their own. Like all aspects of antitumor immune responses, ICD outcomes are generally limited by the immunosuppressive mechanisms active in the tumor, such as innate and adaptive immune checkpoints including CD47, PD-1, and PDL-1—all of which limit the antitumor immune priming and thereby result in sub-optimal therapeutic outcomes.

Thus, there is a critical need to develop complementary treatment approaches that improve local and systemic ICD effects for robust immune effect and tumor control that overcome the disadvantages and defects of the prior art. It is to such new and improved compositions, kits, and systems, as well as methods of producing and using same, that the present disclosure is directed.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1 illustrates that the combination of focused ultrasound (FUS) with calreticulin-nanoparticles (CRT-NPs) therapy increased the CRT expression and CRT/CD47 ratio. (A) Characterization of CRT-NP using gel retardation assay indicated complete encapsulation of CRT plasmid in the NPs. In contrast, blank NPs and CRT-NPs demonstrated no band. (B) Transmission electron microscopy of CRT-NP demonstrated a typical core-shell morphology with the encapsulated plasmid compared to blank NP Scale bar is 100 nm. (C) Quantification of coumarin labeled CRT-NP uptake using flow cytometry showed efficient uptake from 5-8 h similar to blank NPs. The median fluorescence intensity (MFI) of coumarin reduced at 24 h likely due to NP lysis over time. (D) Fluorescence imaging of B16F10 cells incubated with CRT-NPs (2 μg DNA) showed efficient transfection and protein expression (orange) similar to Lipofectamine™2000 (LF2000) transfection reagent (Life Technologies, Carlsbad, Calif.). (E) Flow cytometric analysis of surface expression of CRT 48 h after CRT-NP transfection (4 μg DNA) is shown in bar graph and histogram plot (n=3). Control (grey peak) indicates non-transfected cells. (F and G) Flow cytometric analysis of surface CRT expression (F) and CRT to CD47 ratio (G) in B16F10 cells transfected with CRT-NP (1 μg DNA) for 40-42 h followed by FUS treatment (n=3). CRT-NP+FUS (CFUS) resulted in the highest CRT expression and CRT to CD47 ratio. (H-I) CFUS enhanced tumor regression compared to CRT-NPs. Mice vaccinated s.c. in the flank with 4×10⁶ B16F10 cells transfected with CRT-NPs±FUS (n=5) showed relatively slower tumor growth in CFUS cohorts than CRT-NP. Data are shown as mean±SEM. Statistics were determined by ANOVA followed by Fisher's LSD without multiple comparisons correction. Differences between control and CRT-NP were analyzed using an unpaired t test. * p<0.05, ** p<0.01.

FIG. 2 illustrates that CRT-NP and FUS local treatment enhanced therapeutic efficacy in vivo and synergized when combined as CFUS. (A) Experimental design to test the efficacy of CFUS against melanoma tumors. (B) Immunofluorescence images of B16F10 melanoma tumor sections showing CRT expression (red) and nuclei (DAPI blue). CFUS significantly enhanced CRT intensity in treated tumors compared to other groups (10× magnification). (C) Growth curves of mice tumors in various experimental groups. CFUS significantly achieved tumor growth delay compared to Control, FUS, and CRT-NPs (n=4-7). (D) Differences in the survival were determined for each group by the Kaplan-Meier method, and the overall P value was calculated by the log-rank test. (E) Representative images of the harvested tumor. (F) Tumor weights at the time of sacrifice showed significant reduction in the overall weight with treatments compared to control. Statistics were determined by ANOVA followed by Fisher's LSD without multiple comparisons correction. * p<0.05, ** p<0.01.

FIG. 3 illustrates that Local CRT-NP and FUS therapy activated antigen presenting cells and induced infiltration of T-cells in the tumor. (A) Percentage of CD45 minus (−) cells (tumor cells and fibroblasts) was decreased with CFUS and CRT-NP therapy compared to untreated control. Infiltration of dendritic cells expressing activation marker namely MHCII was enhanced by monotherapies and CFUS. Histogram plots showed an increase in median fluorescence intensity (MFI) of MHCII expressed on intratumoral dendritic cells for the treatments compared to control. (B) Population of CD3+ cells increased by 2-3-fold for FUS, CRT-NP, and CFUS compared to control. (C) CFUS tumors showed ^{˜}2-fold higher ICAM-1 expression than control. (D) Percentage of macrophages (CD11b+F4/80+) in the tumors were analyzed to determine M1 and M2 subtypes. MHCIIhi CD86+ for M1 showed significant increase with CFUS and MHClllo/neg CD206+ was unaltered for the M2 subtypes in treated tumors. Data are shown as mean±SEM, one-way ANOVA followed by Fisher's LSD without multiple comparisons correction. Differences between control and treatments in C were analyzed using an unpaired t test assuming unequal variance. * p<0.05, ** p<0.01.

FIG. 4 illustrates that CFUS improved the local and systemic anti-tumor immunity in B16F10 melanoma model. (A) Mice were challenged in the contralateral flank with 1×10⁵ B16F10 cells 2 weeks post inoculation of the primary tumor (n=4-5). (B) Tumor volumes at the treated tumor site showing significant regression for CFUS and CRT-NPs despite pressure imposed on the immune system by tumor re-challenge. (C) Number of mice that were tumor free at the distant untreated site. Data are shown as mean±SEM, * p<0.05, ** p<0.01; One-way ANOVA followed by Fisher's LSD without multiple comparisons correction.

FIG. 5 illustrates the evaluation of melanoma specific local and systemic immunity in the draining lymph nodes (dLN) and splenic tissue from the mice sacrificed on the same day post inoculation. (A and B) IFN-γ secreting CD8+ T cells in the dLN after ex-vivo stimulation with TRP-2 melanoma antigen showed 1.5-3-fold increase for CRT-NP, FUS and CFUS, compared to untreated control (n=3). IFN-γ+ CD4+ T cells did not change between the treatments compared to control. (C and D) IFN-γ producing CD4+ T cells in the spleen after TRP-2 stimulation were enhanced by CFUS compared to the other groups. IFN-γ+ CD8+ T cells were not altered in the treatment groups (n=3-4). (E) Frequency of M1 macrophages in the spleen was increased by ^{˜}2-fold for CFUS compared to FUS, CRT-NP and control. (F-G) M2 macrophages decreased with CFUS therapy resulting in higher M1 to M2 ratio in the spleen than other cohorts. (H) Weights of the spleen from mice were significantly lowered for the various treatment groups compared to control. Data are shown as mean±SEM. Statistics were determined by ANOVA followed by Fisher's LSD without multiple comparisons correction. Differences between control and treatments in E were analyzed using an unpaired t test assuming unequal variance * p<0.05, ** p<0.01.

FIG. 6 illustrates that CFUS enhanced granzyme-B expression on tumor infiltrating T cells. (A and B) CFUS treated tumors showed a higher percentage of granzyme B+ CD8 and CD4 T cell population than other groups (n=4-7). (C and D) Calculated ratios of % granzyme B+ T cells to Foxp3+ CD4+ Tregs (MFI) were the highest for CFUS tumors (n=4-7). (E) Intratumoral cytokines measured by ELISA. IL-1β was not altered in the treated tumors (n=3). CFUS treatment resulted in a significant increase in TNF-α compared to FUS or CRT-NP alone (n=7-8). Data are shown as mean±SEM, * p<0.05, ** p<0.01; One-way ANOVA followed by Fisher's LSD without multiple comparisons correction.

FIG. 7 illustrates that CRT-based ICD therapy modulated checkpoint markers in tumors and T cells. The surface expression of PD-L1 and PD-1 in treated tumors was analyzed using flow cytometry and western blot and shown as mean±SEM. (A) Percentage of PD-L1+ TILs were significantly enhanced by CFUS and CRT-NP (n=3-4). (B) Frequency of PD-L1+ tumor cells did not change with treatments. (C) Representative western blots of PD-L1 from crude membrane fractions of tumors. Na+/K+ ATPase was used as a loading control (n=4-7). (D) PD-1 expression on CD3+ CD8+ T cells represented as median fluorescence intensity (MFI). Histogram plots indicate the difference in PD-1 MFI among groups. (E) The relationship between the PD-1+ CD8+ T cells and Granzyme B+ CD8+ T cells showed correlation in responding mice. Pearson's r=0.644, p<0.01. Statistics were determined by ANOVA followed by Fisher's LSD without multiple comparisons correction. * p<0.05, ** p<0.01.

FIG. 8 illustrates a dry type FUS system for dog treatment. (a) Experimental set-up for image guided FUS of oral melanoma in intubated dog lying on right side; (b) planning and targeting of FUS; and (c) FUS tumor heating profile (temperature±standard deviation (SD)), measured in oral tumor using fiber-optic sensors. Temperature induction is rapid and returns to normal quickly.

FIG. 9 shows typical location, metastatic feature, and histopathology of oral melanoma in dog. (a) Malignant melanoma between the right canine and right first molar; (b) Confirmation of oral melanoma by CT; (c) Lung metastasis; (d) Moderately sized, spindle to polygonal neoplasm cells arranged in closely packed, small packets and large nests. Small to moderate amounts of dark brown granular cytoplasm with round to oval nuclei with finely stripped chromatin and small distinct solitary basophilic nucleoli.

FIG. 10 shows (a) the oral mass on day 0, (b) FUS ultrasound planning images, and (c) a dramatic remission of benign mass following treatment on day 21; (d) Incorporation of sector-vortex lens in the HIFU transducer; (e) Normalized contour plots of the spatial intensity distribution parallel to the face of the transducer (x-y position); (f) Histopathology of biopsied tissues showed perivascular infiltration of immune cells with no evidence of tumor cells.

### DETAILED DESCRIPTION

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed inventive concept(s) shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses and chemical analyses.

All patents, published patent applications, and non-patent publications mentioned in the specification are indicative of the level of skill of those skilled in the art to which this presently disclosed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions and/or methods disclosed herein can be made and executed without undue experimentation in light of the present disclosure. While the compositions and methods of the inventive concept(s) have been described in terms of particular embodiments, it will be apparent to those of skill in the art that variations may be applied to the compositions and/or methods and in the steps or in the sequence of steps of the methods described herein without departing from the concept, spirit, and scope of the inventive concept(s). All such similar substitutions and modifications apparent to those skilled in the art are deemed to be within the spirit, scope, and concept of the inventive concept(s) as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. For example, the term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

The term “polypeptide” as used herein will be understood to refer to a polymer of amino acids. The polymer may include d-, I-, or artificial variants of amino acids. In addition, the term “polypeptide” will be understood to include peptides, proteins, and glycoproteins.

The term “polynucleotide” as used herein will be understood to refer to a polymer of two or more nucleotides. Nucleotides, as used herein, will be understood to include deoxyribose nucleotides and/or ribose nucleotides, as well as artificial variants thereof. The term polynucleotide also includes single-stranded and double-stranded molecules.

The terms “analog” or “variant” as used herein will be understood to refer to a variation of the normal or standard form or the wild-type form of molecules. For polypeptides or polynucleotides, an analog may be a variant (polymorphism), a mutant, and/or a naturally or artificially chemically modified version of the wild-type polynucleotide (including combinations of the above). Such analogs may have higher, full, intermediate, or lower activity than the normal form of the molecule, or no activity at all. Alternatively and/or in addition thereto, for a chemical, an analog may be any structure that has the desired functionalities (including alterations or substitutions in the core moiety), even if comprised of different atoms or isomeric arrangements.

As used herein, the phrases “associated with” and “coupled to” include both direct association/binding of two moieties to one another as well as indirect association/binding of two moieties to one another. Non-limiting examples of associations/couplings include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety, for example.

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

The term “pharmaceutically acceptable” refers to compounds and compositions which are suitable for administration to humans and/or animals without undue adverse side effects such as (but not limited to) toxicity, irritation, and/or allergic response commensurate with a reasonable benefit/risk ratio.

The term “patient” as used herein includes human and veterinary subjects. “Mammal” for purposes of treatment refers to any animal classified as a mammal, including (but not limited to) humans, domestic and farm animals, nonhuman primates, and any other animal that has mammary tissue.

The term “treatment” refers to both therapeutic treatment and prophylactic or preventative measures. Those in need of treatment include, but are not limited to, individuals already having a particular condition/disease/infection as well as individuals who are at risk of acquiring a particular condition/disease/infection (e.g., those needing prophylactic/preventative measures). The term “treating” refers to administering an agent to a patient for therapeutic and/or prophylactic/preventative purposes.

A “therapeutic composition” or “pharmaceutical composition” refers to an agent that may be administered in vivo to bring about a therapeutic and/or prophylactic/preventative effect.

Administering a therapeutically effective amount or prophylactically effective amount is intended to provide a therapeutic benefit in the treatment, prevention, and/or management of a disease, condition, and/or infection. The specific amount that is therapeutically effective can be readily determined by the ordinary medical practitioner, and can vary depending on factors known in the art, such as (but not limited to) the type of condition/disease/infection, the patient's history and age, the stage of the condition/disease/infection, and the co-administration of other agents.

The term “effective amount” refers to an amount of a biologically active molecule or conjugate or derivative thereof sufficient to exhibit a detectable therapeutic effect without undue adverse side effects (such as (but not limited to) toxicity, irritation, and allergic response) commensurate with a reasonable benefit/risk ratio when used in the manner of the inventive concept(s). The therapeutic effect may include, for example but not by way of limitation, preventing, inhibiting, or reducing the occurrence of infection by or growth of microbes and/or opportunistic infections. The effective amount for a subject will depend upon the type of subject, the subject's size and health, the nature and severity of the condition/disease/infection to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

As used herein, the term “concurrent therapy” is used interchangeably with the terms “combination therapy” and “adjunct therapy,” and will be understood to mean that the patient in need of treatment is treated or given another drug for the disease/infection in conjunction with the pharmaceutical compositions of the present disclosure. This concurrent therapy can be sequential therapy, where the patient is treated first with one pharmaceutical composition and then the other pharmaceutical composition, or the two pharmaceutical compositions are given simultaneously.

The terms “administration” and “administering,” as used herein, will be understood to include all routes of administration known in the art, including but not limited to, oral, topical, transdermal, parenteral, subcutaneous, intranasal, mucosal, intramuscular, intraperitoneal, intravitreal, and intravenous routes, and including both local and systemic applications. In addition, the compositions of the present disclosure (and/or the methods of administration of same) may be designed to provide delayed, controlled, or sustained release using formulation techniques which are well known in the art.

The terms “room temperature” and “ambient temperature” are used herein interchangeably and refer to a temperature in a range of from about 18° C. to about 28° C.

Advanced stage IV cancer is a highly lethal form of cancer that is typically refractory to chemotherapy, radiation, and surgery. Fortunately, the advent of immunotherapeutics as the fourth treatment approach over the last few years is beginning to change the landscape. In this approach, the drug agents stimulate the patient's immune system to fight off cancer cells. However, even with this approach, the response rate in patients remains below 40%.

An effective tumor immune therapy must meet certain criteria; it should break immune tolerance, prime tumors in a way that induces its local and systemic clearance, and be nontoxic. To meet these criteria, the crucial aspect appears to be “immunogenic cell death” (ICD) that occurs when cells die in a manner that stimulates the immune system. Thus, innovative technologies that modulate the tumor in a way that creates a more immunogenic environment is an urgent need for faster clearance of solid tumors and for enhancing the efficacy of existing drugs.

The present disclosure proposes a device-directed approach of immune stimulation for treatment of (for example, but not by way of limitation) established tumors or as a prophylaxis therapy to prevent (for example, but not by way of limitation) initial cancer or cancer recurrence. In certain non-limiting embodiments, ICD stimulating nanoparticles were created that synergistically enhance the efficacy of cancer therapy in combination with a focused ultrasound therapy device. The nanoparticle alone was also efficacious, demonstrating that the formulation approach can also improve the therapeutic efficacy of currently available immunotherapeutics in clinical use. Thus, the focused ultrasound activated nanoparticle technology of the present disclosure can optimize therapeutic outcomes in advanced stage cancer patients. Further, the technology of the present disclosure can prevent cancer recurrence in successfully treated cancer patients.

Certain non-limiting embodiments of the present disclosure are directed to compositions, systems, and methods related to the use of gene delivery nanoparticles (NPs) for the intratumoral expression of an ICD agent, calreticulin (CRT), which can be utilized alone or further be combined with other compositions or technologies (such as, but not limited to, focused ultrasound (FUS)) to synergistically activate antitumor immune effects against cancer cells.

Further non-limiting embodiments of the present disclosure are directed to compositions, systems, and methods for pretreating patient's cancer cells in vitro by transfecting said cancer cells with nanoparticles containing a CRT gene (CRT-NPs), or by treating said cancer cells with focused ultrasound (FUS), or by combining CRT-NP transfection with FUS treatment for enhanced transfection of said cancer cells. Thus, the compositions, systems, and methods of the present disclosure can be used to generate pretreated cancer cells for in vivo vaccination of a patient's tumor.

The use of immunotherapy is effective against a broad spectrum of cancer types; however, only a minority of patients show a durable response (<30%). The challenge is attributed to heterogeneity in immune suppression in tumors with similar histological features, variability in the frequency of mutations burden, adverse toxicity profile, age-related senescence, and disordered and immunosuppressive tumor microenvironment. Recent studies indicate that the removal of local tumor immunosuppression with in-situ vaccination (ISV) can provide twin benefits of treating the tumor itself as well as using the tumor as the antigen source for a systemic response while applying some sort of vaccine adjuvant. This has been done with oncolytic and plant viruses, lipid-coated cisplatin nanoparticles (LPC) and CpG-encapsulated liposomes (CpG-Lipo), and a-gal glycolipids etc., and a recent version of the oncolytic virus, T-vec. It is important to note that the local immunotherapy strategy of ISV does not preclude other immunotherapies applied systemically, such as checkpoint blockade, that are clinically available.

Currently, most ISV strategies are focusing on delivering a vaccine adjuvant of some sort into the tumor, but another aspect of immune recognition that can fundamentally alter the immune biology in a tumor is the induction of an immunogenic cell death (ICD) using nanoparticles. The present disclosure focused, in certain non-limiting embodiments, on creating novel approaches of delivering into cells genes or other therapeutic agents (i.e., proteins) that express ICD markers (e.g. Calreticulin or CRT). CRT is an abundant 46 kDa Ca²⁺-binding protein in the endoplasmic reticulum (ER). CRT can enhance immune response following exogenous administrations using adenoviral vector or combination with photodynamic therapy. The amino acid sequences in the CRT protein are homologous and conserved between mice and humans, and it can be expressed in a pre-apoptotic cell for recognition by antigen presenting cells (APCs) to aid in cancer cell phagocytosis. Currently, CRT expression with adenovirus (AV)/lipofectamine vectors has several limitations. AVs can be limited by the size of the genetic material they can deliver, have high immunogenicity, and are potentially oncogenic, thereby making the clinical translation challenging.

As an alternative, lipid-based nanoparticles (NP) were developed herein to achieve high transfection efficiency of CRT to complement the FUS therapy. While the idea of lipid-based agents for gene transfection has been shown previously, the novel approaches of the present disclosure demonstrate: 1) the utilization of these materials for CRT delivery, and 2) the synergistic activation of clinical response in combination with focused ultrasound.

Certain non-limiting embodiments of the present disclosure are directed to a composition that comprises a nanoparticle comprising a calreticulin gene or a fragment or variant thereof. The term “calreticulin” may be used interchangeably with the terms CRT, CALR, calregulin, CRP55, CaBP3, calsequestrin-like protein, endoplasmic reticulum resident protein 60 (ERp60), Sicca Syndrome Antigen A (SSA), or CRTC.

The calreticulin gene or fragment/variant thereof may possess any nucleotide sequence disclosed or otherwise contemplated herein. For example, the calreticulin gene/fragment variant may possess the nucleotide sequence of SEQ ID NO:1, or may be at least about 50% identical to SEQ ID NO:1 (such as, but not limited to, at least about 55%, at least about 60%, at least about 65%, at least about 70%, at least about 75%, at least about 80%, at least about 85%, at least about 90%, at least about 95%, or at least about 98% identical to the sequence of SEQ ID NO:1). Alternatively (and/or in addition thereto), the calreticulin gene/fragment variant thereof may be able to hybridize to a complement of SEQ ID NO:1.

In another non-limiting embodiment, the calreticulin gene/fragment/variant may be encoded by at least a portion of the amino acid sequence of SEQ ID NO:2, or may be encoded by a sequence that is at least about 50% identical to the amino acid sequence of SEQ ID NO:2 (such as, but not limited to, at least about 55%, at least about 60%, at least about 65%, at least about 70%, at least about 75%, at least about 80%, at least about 85%, at least about 90%, at least about 95%, or at least about 98% identical to the amino acid sequence of SEQ ID NO:2).

In another non-limiting embodiment, the calreticulin gene/fragment/variant may have a nucleotide sequence that varies from the nucleotide sequence of SEQ ID NO:1 by less than about 200 nucleotides, such as, but not limited to, by less than about 190 nucleotides, by less than about 180 nucleotides, by less than about 170 nucleotides, by less than about 160 nucleotides, by less than about 150 nucleotides, by less than about 140 nucleotides, by less than about 130 nucleotides, by less than about 120 nucleotides, by less than about 110 nucleotides, by less than about 100 nucleotides, by less than about 90 nucleotides, by less than about 80 nucleotides, by less than about 70 nucleotides, by less than about 60 nucleotides, by less than about 50 nucleotides, by less than about 45 nucleotides, by less than about 40 nucleotides, by less than about 35 nucleotides, by less than about 30 nucleotides, by less than about 25 nucleotides, by less than about 20 nucleotides, by less than about 15 nucleotides, by less than about 10 nucleotides, or by less than about 5 nucleotides.

In another non-limiting embodiment, the calreticulin gene/fragment/variant may have a nucleotide sequence encoded by an amino acid sequence that differs from the amino acid sequence of SEQ ID NO:2 by less than about 75 amino acids, such as, but not limited to, by less than about 70 amino acids, by less than about 65 amino acids, by less than about 60 amino acids, by less than about 55 amino acids, by less than about 50 amino acids, by less than about 45 amino acids, by less than about 40 amino acids, by less than about 35 amino acids, by less than about 30 amino acids, by less than about 25 amino acids, by less than about 24 amino acids, by less than about 23 amino acids, by less than about 22 amino acids, by less than about 21 amino acids, by less than about 20 amino acids, by less than about 19 amino acids, by less than about 18 amino acids, by less than about 17 amino acids, by less than about 16 amino acids, by less than about 15 amino acids, by less than about 14 amino acids, by less than about 13 amino acids, by less than about 12 amino acids, by less than about 11 amino acids, by less than about 10 amino acids, by less than about 9 amino acids, by less than about 8 amino acids, by less than about 7 amino acids, by less than about 6 amino acids, by less than about 5 amino acids, by less than about 4 amino acids, by less than about 3 amino acids, by less than about 2 amino acids, or by less than about 1 amino acid.

When the composition comprises a fragment of a calreticulin gene, the fragment may comprise at least about 700 nucleotides of the calreticulin gene, such as but not limited to, at least about 750 nucleotides, at least about 800 nucleotides, at least about 850 nucleotides, at least about 900 nucleotides, at least about 950 nucleotides, at least about 1000 nucleotides, at least about 1025 nucleotides, at least about 1050 nucleotides, at least about 1075 nucleotides, at least about 1100 nucleotides, at least about 1110 nucleotides, at least about 1120 nucleotides, at least about 1130 nucleotides, at least about 1135 nucleotides, at least about 1140 nucleotides, at least about 1145 nucleotides, at least about 1150 nucleotides, at least about 1155 nucleotides, at least about 1160 nucleotides, at least about 1165 nucleotides, at least about 1170 nucleotides, at least about 1175 nucleotides, at least about 1180 nucleotides, at least about 1185 nucleotides, at least about 1190 nucleotides, at least about 1200 nucleotides, at least about 1205 nucleotides, at least about 1210 nucleotides, at least about 1215 nucleotides, at least about 1220 nucleotides, at least about 1225 nucleotides, at least about 1250 nucleotides, at least about 1251 nucleotides, at least about 1252 nucleotides, or at least about 1253 nucleotides of the calreticulin gene.

In addition, when the composition comprises a fragment of a calreticulin gene, the fragment may comprise all but about 200 nucleotides of the calreticulin gene, such as but not limited to, all but about 190 nucleotides, all but about 180 nucleotides, all but about 170 nucleotides, all but about 160 nucleotides, all but about 150 nucleotides, all but about 140 nucleotides, all but about 130 nucleotides, all but about 120 nucleotides, all but about 110 nucleotides, all but about 100 nucleotides, all but about 90 nucleotides, all but about 80 nucleotides, all but about 70 nucleotides, all but about 60 nucleotides, all but about 55 nucleotides, all but about 50 nucleotides, all but about 45 nucleotides, all but about 40 nucleotides, all but about 35 nucleotides, all but about 30 nucleotides, all but about 25 nucleotides, all but about 20 nucleotide, all but about 15 nucleotides, all but about 10 nucleotides, or all but about 5 nucleotides of the calreticulin gene.

Further, when the composition comprises a fragment of a calreticulin gene, the fragment may encode at least about 300 amino acids of SEQ ID NO:2, such as at least about 310 amino acids, at least about 320 amino acids, at least about 330 amino acids, at least about 340 amino acids, at least about 350 amino acids, at least about 360 amino acids, at least about 370 amino acids, at least about 375 amino acids, at least about 380 amino acids, at least about 385 amino acids, at least about 390 amino acids, at least about 395 amino acids, at least about 400 amino acids, at least about 401 amino acids, at least about 402 amino acids, at least about 403 amino acids, at least about 404 amino acids, at least about 405 amino acids, at least about 406 amino acids, at least about 407 amino acids, at least about 408 amino acids, at least about 409 amino acids, at least about 410 amino acids, at least about 411 amino acids, at least about 412 amino acids, at least about 413 amino acids, at least about 414 amino acids, at least about 415 amino acids, or at least about 416 amino acids of the calreticulin amino acid sequence of SEQ ID NO:2.

The activities of calreticulin have been well studied, and various fragments and variants thereof that retain the activities relied upon in the present disclosure are well known in the art and readily available. See, for example, U.S. Pat. No. 7,488,711; Korbelik et al. (Front Oncol (2015) 5:Article 15 (pages 1-7)); Holmstrom et al. (Leukemia (2018) 32:429-437); Wang et al. (Intl Cancer (2012) 130:2892-2902); Molina et al. (Mol Biochem Parasitol (2005) 140(2):133-40); Cheng et al. (J Clin Invest (2001) 108(5):669-678); and Hong et al. (J Immuno) (2010) 185(8):4561-4569); the entire contents of each of which are hereby expressly incorporated herein by reference.

Thus, it is well within the purview of a person having ordinary skill in the art to truncate the calreticulin sequence at various positions to produce calreticulin fragments that retain the desired activity of native calreticulin. Likewise, it is well within the purview of a person having ordinary skill in the art to modify or mutate certain residues of the calreticulin sequence to produce calreticulin variants that retain the desired activity of native calreticulin. Thus, no further description regarding calreticulin fragments or variants is deemed necessary to identify and practice the full scope of the present disclosure.

Any types of nanoparticles known in the art or otherwise contemplated herein may be utilized in accordance with the present disclosure. For example, but not by way of limitation, the nanoparticles may be liposomes; polymeric, hybrid, iron oxide, chitosan, inorganic, or organic NPs; or oncolytic viruses or viral particles.

In certain particular (but non-limiting) embodiments, the particles are non-viral.

In certain non-limiting embodiments, the nanoparticle comprises a liposome. In a particular but non-limiting embodiment, the liposome is a cationic liposome.

The nanoparticles may be formed by any methods and from any substances known in the art or otherwise contemplated herein. Non-limiting examples of materials from which the nanoparticles can be formed include DOTAP (1,2-dioleoyl-3-trimethylammonium-propane), cholesterol, cationic and anionic lipids, polymers, and any combinations thereof. In a particular (but non-limiting) embodiment, the nanoparticle is a liposome that comprises DOTAP and cholesterol.

The nanoparticles may be provided with any structural characteristics that allow the compositions disclosed herein to function in accordance with the present disclosure. For example, but not by way of limitation, the nanoparticle may have a hydrodynamic diameter in a range of from about 200 nm to about 300 nm, such as, but not limited to, a range of from about 240 nm to about 270 nm. Alternatively and/or in addition thereto, the nanoparticle may have a Zeta potential in a range of from about +1 mV to about +20 mV and a polydispersity index of less than about 0.3.

The composition may be substantially stable at room temperature (i.e., a temperature in a range of from about 20° C. to about 25° C.) for a desirable period of time. For example (but not by way of limitation), the composition may be substantially stable at room temperature for at least about 7 days, such as, but not limited to, at least about 14 days, at least about 21 days, at least about 30 days, at least about 45 days, at least about 2 months, at least about 3 months, at least about 4 months, at least about 5 months, at least about 6 months, or more.

Certain non-limiting embodiments of the present disclosure are directed to a pharmaceutical composition that comprises any of the nanoparticle-containing compositions disclosed or otherwise contemplated herein in combination with a pharmaceutically acceptable carrier or excipient.

The pharmaceutical composition may be formulated for any mode of administration that allows the nanoparticle-containing composition to function in accordance with the present disclosure. For example, the pharmaceutical compositions of the present disclosure may be formulated for administration by any of the many suitable means described herein and/or which are well known to those of skill in the art, including but not limited to: by injection, inhalation, oral, intravaginal, intranasal, rectal, or intradermal administration; by ingestion of a food or probiotic product containing the virus; by topical administration, such as (but not limited to) as eye drops, sprays, etc.; by implantation in the form of an implant; and the like.

In a particular (but non-limiting) embodiment, the pharmaceutical composition may be formulated for injection into a patient.

Certain non-limiting embodiments of the present disclosure are directed to a kit that comprises at least one of any of the nanoparticle-containing compositions disclosed or otherwise contemplated herein and/or any of the pharmaceutical compositions disclosed or otherwise contemplated herein.

The compositions present in the kits may be provided in any form that allows them to function in accordance with the present disclosure. For example, but not by way of limitation, each of the reagents may be disposed in bulk and/or single aliquot form within the kit. Alternatively, in a particular (but non-limiting) embodiment, one or more of the compositions may be disposed in the kit in the form of multiple single aliquots of the same or different compositions.

In addition to the compositions described in detail herein above, the kits may further contain other reagent(s)/composition(s)/device(s) for use in the methods described or otherwise contemplated herein. The nature of these additional reagent(s)/composition(s)/device(s) will depend upon the particular treatment(s) being used, and identification thereof is well within the skill of one of ordinary skill in the art in light of the present disclosure; therefore, no further description thereof is deemed necessary. Also, the reagent(s)/composition(s)/device(s) present in the kits may each be in separate containers/compartments, or various reagents/compositions/devices can be combined in one or more containers/compartments, depending on the cross-reactivity and stability of the reagents/compositions/devices.

In certain particular (but non-limiting) embodiments, the kit can further contain one or more of a composition containing a PD-1 inhibitor (such as, but not limited to, an anti-PD-1 antibody); a composition containing a PDL-1 inhibitor (such as, but not limited to, an anti-PDL-1 antibody); a composition containing a CD47 blocking antibody; and/or an instrument for performing focused ultrasound, each as described in detail herein.

The relative amounts of the various compositions/reagents present in the kits can vary widely to provide for concentrations of the compositions/reagents that substantially optimize the treatment methods. Under certain circumstances, one or more of the compositions/reagents in the kit can be provided as a dry powder, such as a lyophilized powder, and the kit may further include excipient(s) for dissolution of the dried reagents; in this manner, a solution having the appropriate concentrations of the compositions/reagents for performing a method in accordance with the present disclosure can be obtained from these components. In addition, the kit can further include a set of written instructions explaining how to use the kit. A kit of this nature can be used in any of the methods described or otherwise contemplated herein.

Certain non-limiting embodiments of the present disclosure are directed to a system for treatment of cancer or reduction in the occurrence of cancer, wherein the system comprises any of the nanoparticle-containing compositions disclosed or otherwise contemplated herein in combination with an instrument for performing focused ultrasound (FUS).

FUS treatment parameters can be adjusted in accordance to existing protocols and optimized based on requirements of a treatment plan. In a non-limiting example, FUS parameters may be selected from short-duration hyperthermia, long-duration hyperthermia, histotripsy, low intensity focused ultrasound, ablation, and combinations thereof. In yet another non-limiting example, variable FUS treatment parameters include pulse repetition frequencies (PRF), duty cycle, and acoustic power. In certain particular (but non-limiting) embodiments, FUS pulse repetition frequency can be in a range of from about 1 Hz to about 100 Hz. In another non-limiting embodiment, FUS duty cycle is in a range of from about 0.01% to about 100%. In yet another non-limiting embodiment, acoustic power is in a range of from about 1 W to about 600 W.

By changing FUS treatment parameters, optimal therapeutic benefit can be achieved.

In a particular (but non-limiting) embodiment, the focused ultrasound instrument has a target temperature in a range of from about 40° C. to about 60° C.

In certain particular (but non-limiting) embodiments, the system may further contain one or more of a composition containing a PD-1 inhibitor (such as, but not limited to, an anti-PD-1 antibody); a composition containing a PDL-1 inhibitor (such as, but not limited to, an anti-PDL-1 antibody); a composition containing a CD47 inhibitor (such as, but not limited to, a CD47 blocking antibody); and/or other immunomodulatory agents.

Certain non-limiting embodiments of the present disclosure are directed to a method that comprises the step of administering any of the nanoparticle compositions and/or pharmaceutical compositions disclosed or otherwise contemplated herein to at least a portion of a patient in need thereof.

Certain non-limiting embodiments of the present disclosure are directed to a method that comprises the steps of: (i) administering any of the nanoparticle compositions and/or pharmaceutical compositions disclosed or otherwise contemplated herein to at least a portion of a patient in need thereof; and (ii) administering focused ultrasound to the patient so as to heat at least a portion of a particular tissue; and wherein steps (i) and (ii) are performed simultaneously or wholly or partially sequentially; and wherein the combination of steps (i) and (ii) induces immunogenic cell death within the particular tissue. In certain non-limiting embodiments, the particular tissue is a tumor, surrounding tissue of a tumor, tissue predisposed to developing a tumor, etc., or any combination thereof. In certain non-limiting embodiments, the method is an in vivo cancer treatment or cancer prevention method.

In certain non-limiting embodiments, the method disclosed herein may be utilized in combination with one or more additional treatment steps, such as (but not limited to) administration of radiation (such as, but not limited to, photons, laser, protons, carbon beam, etc.) and/or surgery to remove a tumor. That is, a clinician may surgically remove a primary tumor, then step (i) alone or steps (i) and (ii) are performed, by administering the nanoparticle-containing composition and/or the focused ultrasound to the area surrounding where the tumor was surgically removed (i.e., the potential “tumor margins”). In addition, the methods disclosed herein can be utilized for both active treatment of existing cancer as well as prophylaxis (i.e., prevention of cancer in high-risk patients, reducing the chance of cancer reoccurrence among patients in remission, prevention of metastasis in patients diagnosed and successfully treated at an early stage of cancer, etc.). As such, the methods disclosed herein may serve as an adjuvant therapy for existing standards of care.

In a particular (but non-limiting) embodiment, at least a portion of the particular tissue is heated to a target temperature in a range of from about 40° C. to about 60° C.

In a particular (but non-limiting) embodiment, short-duration hyperthermia, long-duration hyperthermia, or ablation is induced by the administration of focused ultrasound to the patient.

In a particular (but non-limiting) embodiment, the focused ultrasound emits a pulse repetition frequency in a range of from about 1 Hz to about 100 Hz, a duty cycle in a range of from about 10% to about 100%, and/or an acoustic power in a range of from about 1 W to about 100 W.

In a particular (but non-limiting) embodiment, steps (i) and (ii) are performed wholly or partially sequentially, and step (i) is performed prior to step (ii).

In a particular (but non-limiting) embodiment, steps (i) and (ii) are performed wholly or partially sequentially, and step (ii) is performed prior to step (i).

In a particular (but non-limiting) embodiment, steps (i) and (ii) are performed substantially simultaneously.

In a particular (but non-limiting) embodiment, the method further comprises the step of repeating steps (i) and (ii) one or more times to induce immunogenic cell death within the particular tissue.

The pharmaceutical compositions of the present disclosure may be administered by any of the many suitable means described herein and/or which are well known to those of skill in the art, including but not limited to: by injection, inhalation, oral, intravaginal, intranasal, rectal, or intradermal administration; by ingestion of a food or probiotic product containing the virus; by topical administration, such as (but not limited to) as eye drops, sprays, etc.; by implantation of an implant; and the like. In particular (but non-limiting) embodiments, the mode of administration is by injection and/or inhalation. For example (but not by way of limitation), the route of delivery of the composition may be intratumoral, intramuscular, intravenous, intradermal, etc.

One or more than one route of administration can be employed either simultaneously or partially or wholly sequentially, i.e., prime boost vaccine regimens are also contemplated. Such prime boost vaccine regimens typically involve repeated vaccine administrations at preselected intervals, such as (but not limited to) every month, every six weeks, every two months, every three months, every six months, every year, every 18 months, every two years, every three years, every four years, or at longer intervals, e.g., every five or ten years, etc. Those of skill in the art are well acquainted with the planning, implementation, and assessment of such vaccine strategies, and therefore no further discussion thereof is required.

The pharmaceutical compositions may be administered in conjunction with other treatment modalities. In some embodiments, such modalities may include (but are not limited to) various substances that boost the immune system, various chemotherapeutic agents, vitamins, anti-allergy agents, anti-inflammatory agents, etc. In other embodiments, other antigenic agents (e.g., other vaccines or vaccinogens), may be advantageously administered or co-administered with the pharmaceutical compositions disclosed or otherwise contemplated herein. For example (but not by way of limitation), in some cases it may be desirable to combine the method of the present disclosure with known checkpoint inhibitors that block PD-1, PD-L1, and/or CD47. When multiple immunogenic compositions/vaccines are to be administered together, the immunogenic compositions/vaccine agents may be combined in a single pharmaceutical composition. Alternatively (and/or in addition thereto), the multiple immunogenic compositions/vaccines may be administered separately but over a short time interval, e.g., at a single visit at a doctor's office or clinic, etc.

In a particular (but non-limiting) embodiment, the method further comprises the step of: (iii) administering an immunomodulatory agent to the patient. In certain non-limiting embodiments, the immunomodulatory agent is selected from a PD-1 inhibitor, a PDL-1 inhibitor, a CD40 inhibitor, an OX40 inhibitor, and/or a CD47 inhibitor For example, step (iii) may be performed prior to steps (i) and (ii), or step (iii) may be performed after steps (i) and (ii), or step (iii) may be performed simultaneously with one or both of steps (i) and (ii), or step (iii) may be performed wholly or partially sequentially with steps (i) and (ii) (including before or after step (i), before or after step (iii), and/or sequentially with one step and before or after the other step). When steps (i) and (iii) are performed simultaneously, the two compositions may be combined within a single pharmaceutical composition, or the two compositions may be present separately and simply administered simultaneously.

In a particular (but non-limiting) embodiment, the inhibitor(s) present in the composition utilized in step (iii) may be an antibody; for example (but not by way of limitation), the PD-1, PDL-1, or CD47 inhibitor may be an anti-PD-1 antibody, an anti-PDL-1 antibody, or a CD47 antibody, respectively.

In a further non-limiting embodiment, the method further comprises the step of: (iv) administering a CD47 blocking antibody to the patient. For example, For example, step (iv) may be performed prior to steps (i), (ii), and (iii); or step (iv) may be performed after steps (i), (ii), and (iii); or step (iv) may be performed concurrently with one or more of steps (i), (ii), and (iii), or step (iv) may be performed in between any of the other two steps.

In addition, steps (iii) and (iv) may be performed in the same method, or a method may only include one of step (iii) or (iv).

Subjects to which the compositions disclosed or otherwise contemplated herein may be administered may be mammals and are frequently humans, particularly human cancer patients or patients at risk of developing cancer. However, this need not always be the case. Veterinary uses of the pharmaceutical compositions and methods disclosed or otherwise contemplated herein are also contemplated, e.g., for companion pets, or for animals that are of commercial value, e.g., as a food source, or for any other animal, etc.

The nanoparticle-containing compositions disclosed or otherwise contemplated herein may be utilized to treat different cancer types, including (but not limited to) melanoma.

### EXAMPLES

Examples are provided hereinbelow. However, the present disclosure is to be understood to not be limited in its application to the specific experimentation, results, and laboratory procedures disclosed herein. Rather, the Examples are simply provided as one of various embodiments and are meant to be exemplary, not exhaustive.

### Example 1

Calreticulin is a multifunctional calcium binding chaperone protein that plays a major role in maintaining calcium stores in the lumen of the endoplasmic reticulum (ER). Calreticulin has also been shown to modulate transcriptional activity by interacting with nuclear hormone receptors, and the sub-cellular localization of calreticulin can aid in the modulation of various cellular functions, including cell death and immune responses.

CRT increases the infiltration of lymphocytes into tumors by regulating the vascular endothelial expression of adhesion molecules, thus enhancing the immune response in an immunosuppressive microenvironment. This characteristic can greatly enhance the antitumor efficacy of an immunotherapy. With its multiple antitumor mechanisms, CRT represents a therapeutic agent for cancer therapy.

CRT proteins enhance the phagocytosis and immunogenic recognition of dying cancer cells by antigen presenting cells (APCs) and improve interactions with tumor infiltrating leukocytes by modulation ICAM-1 and VCAM-1 on tumor endothelial cells. Additionally, tumor-associated macrophages expressing activated CRT on cell surfaces exhibit efficient phagocytosis of cancerous cells. Although able to contribute to the needed immune stimulation, CRT productions are often inconsistent in most solid tumors and by themselves generally are not sufficient to generate an effective antitumor immune response. Thus, there is a critical need to develop novel approaches that improve local CRT-based ICD outcomes in immunosuppressive tumors.

The objective of this Example was to develop novel calreticulin nanoparticles (CRT-NPs) and combine CRT-NPs with focused ultrasound (FUS) for ICD based immunomodulation in melanoma. The CRT-NP of the present disclosure is a biocompatible liposome-based gene delivery agent. Unlike viral vectors that risk mutagenic integration with host cells, the liposomes are a promising yet safer gene-drug delivery technology. FUS is an extracorporeal treatment device that delivers focused sound waves non-invasively, providing a powerful tool for clinical administration of anatomically-specified thermal effect in soft tissues. The inventors and others have shown that the FUS-induced thermal effect modifies the tumor microenvironment to impart several benefits, including (but not limited to) enhanced response to chemotherapy, tumor antigen release, expression of heat-shock proteins, upregulation of pro-phagocytic signals such as CRT, and overall tumor immunity stimulation compared to conventional treatment. Based on this premise, it was hypothesized that the direct intratumoral (in-situ) injection of CRT-NP in easily accessible melanoma tumors and combined with FUS heating (42-45° C.) would abrogate the aberrant and tumor-suppressive factors and trigger the clearance of melanoma cells.

To test this hypothesis, the infiltration of activated macrophages and CD8+ T cells, and the expression of innate (CD47) and adaptive markers (PD-1, PD-L1, etc.) were characterized in the immunosuppressive B16F10 model. CD47 membrane protein interacts with receptor signal regulatory protein on immune cells to inhibit phagocytosis by the macrophages. Like CD47, the expression of checkpoints, namely PD-1 on T cells, impairs the interaction of effector T cells and macrophages with tumor cells via the PD-1/PD-L1 axis. The data present in this Example indicates that the FUS and CRT-NP combination transforms melanoma tumor immune microenvironmental factors, aiding immune clearance. Thus, the combinatorial modality of the present disclosure has the potential to ease the clinical translation of ICD approaches in clinics.

Material and Methods

Materials

1,2-dioleoyl-3-trimethylammonium-propane (DOTAP) was purchased from Corden Pharma (Wouburn, Mass.) or Avanti Polar Lipids (Alabaster, Ala.), and Cholesterol was purchased from Calbiochem (San Diego, Calif.). B16F10 murine melanoma cells were obtained from Dr. Mary Jo Turk at Geisel School of Medicine at Dartmouth (Hanover, N.H.). B16F10 cells were cultured in DMEM supplemented with 10% fetal bovine serum (FBS) and 1% streptomycin/penicillin. Plasmids containing CRT genes controlled by the CMV promoter were obtained from Sino Biological (Collegeville, Pa.). Fluorochrome-conjugated monoclonal antibodies (mAbs) for flow cytometry were purchased from BioLegend (San Diego, Calif.) and are listed here: APC anti-CD4 (GK1.5), PE anti-CD3 (145-2C11), PERCP anti-F4/80 (BM8), APC-Cy7 anti-MHCII (M5/114.15.2), APC-CY7 anti-IFN-γ (XMG1.2), APC anti-CD11c (N418), FITC anti-CD45.2 (104), FITC anti-CD86 (GL-1), FITC anti-Granzyme B (GB11), FITC anti-PD-1 (29F.1A1.2), Alexa fluor 700 anti-CD206 (C068C2), PE or APC anti-CD11b (M1/70), FITC anti-Gr-1 (RB6-8C5), Pacific blue anti-ICAM-1 (YN1/1.7.4), PE anti-PDL-1 (10F.9G2), and PE anti-CD47 (miap301). FITC anti-CD8a (53-6.7) and PE anti-Foxp3 (R16-715) were purchased from BD Biosciences (San Jose, Calif.). Alexa fluor 647 anti-CRT (EPR3924) was obtained from Abcam (Cambridge, Mass.).

Synthesis and Characterization of CRT-NP

Full-length clone DNA of human CRT cloned into pCMV3 vector was used (HG13539-ACR, Sino Biological Inc., Wayne, Pa.). For CRT-NP synthesis, a lipid film was hydrated in 10 mM HEPES buffer (pH 7.4) at 55° C., and the lipid suspension was then extruded five times through filters of 200 nm pore size to yield homogeneous liposomes [34]. Next, a one-step method for loading the plasmid was developed by adding the pDNA solution in the liposomes vial (1:10, wt/wt), gently mixing them by pipetting, and incubating at room temperature for 30 min. The resultant CRT-NPs were characterized for plasmid encapsulation by the gel retardation assay in 1% agarose pre-cast gels containing ethidium bromide. A control sample of free pCRT as well as blank NPs were loaded onto the gels and the gels were run at 80 V on a Bio-Rad electrophoresis system. DNA dose was 0.2 μg per lane. Approximately 60 minutes after beginning the run, the gels were observed for plasmid migration. The CRT-NPs were also characterized in physiological buffers at room temperature by size (z-average) and zeta-potential using dynamic light scattering (DLS) with a Brookhaven ZetaPALS instrument (Holtsville, N.Y.). Furthermore, transmission electron microscopy (TEM) as previously published was performed to assess the morphology of CRT-NP using the JEOL JEM-2100 TEM (JEOL USA, Peabody, Mass.).

Assessment of Transfection and Uptake Efficiency of CRT-NPs in Melanoma Cells In Vitro with Fluorescence Microscopy and Flow Cytometry

For in vitro transfection assessment, full-length clone DNA of human CRT with a C terminal OFP Spark tag cloned into pCMV3 vector was used. 1×10⁵ B16F10 cells per well were seeded in 24-well plates for 18-24 h prior to transfection. On the day of transfection, cell culture medium was replaced with serum free medium and the cells were incubated with CRT-NPs (2 μg total DNA per well) at 37° C. and 5% CO₂ for 5 h. Next, the cells were rinsed to remove non-phagocytosed NPs and incubated for an additional 48 h with DMEM containing 10% serum to assess the CRT expression. To compare the transfection efficiency, CRT plasmid was complexed with commercially available Lipofectamine™2000 (LF2000) transfection reagent (Life Technologies, Carlsbad, Calif.) and used as a positive control according to manufacturer's instructions. Fluorescence imaging of CRT was performed with the RFP filter cube (ex/em of 531/593 nm) using Biotek Cytation 5 cell imaging multimode reader (Winooski, Vt.) and the images were acquired using Gen5 Image+software version 3.08.01. Image acquisition and display parameters were kept constant to allow for qualitative comparison. Transfected cells (impermeabilized) were stained with surface targeting Alexa fluor 647 anti-CRT antibody and cell surface expression of CRT was quantified by flow cytometry in an LSRII analyzer (BD Biosciences, Franklin Lakes, N.J., and U.S.A.). In addition, we characterized the CRT-NP uptake efficiencies in B16F10 cells using flow cytometry. Briefly, B16F10 cells were incubated with coumarin dye labeled CRT-NPs for 5, 8, and 24 h, and the mean fluorescence intensity (MFI) was assessed and compared (n=3).

FUS Treatment Methodology for In Vitro and In Vivo Assays

An imaging and therapeutic ultrasound system (Alpinion Medical Systems, Bothell, Wash.) was used for all FUS exposures. The system consists of FUS transducer with a 1.5 MHz central frequency, 45 mm radius, and 64 mm aperture diameter with a central opening of 40 mm in diameter and an automated motion stage to achieve accurate positioning perpendicular to FUS beam axis. FUS treatment parameters used were as follows: 5 Hz frequency, 50% duty cycle, and 6 W power (equivalent to 3.5 W acoustic power). This method achieved a mean target temperature of 42-45° C. at the focus inside the tumor (measured by inserting a fiber optic temperature sensor; Qualitrol, Quebec, Canada). The duration of FUS exposure at the focus was 15 min. For tumor treatments, the center of the tumor was aligned at a fixed focal depth for efficient coverage voxel size (5×5×12 mm) using a sector vortexed lens [24]. As the tumor grew, the focal point was rastered to cover the entire tumor. For in-vitro FUS treatments, the transducer was used without lens to achieve a coverage of 1×1×10 mm. An integrated VIFU-2000 software was used to define target boundary and slice distance in x, y, and z directions for automatic rastering of the transducer for 15 min.

Assessment of CRT and CD47 Expression in B16F10 Cell Membranes Post Treatments, and Evaluation of Tumor Growth In Vivo

5×10⁵ B16F10 cells per well were seeded in 6-well plates 18-24 h prior to transfection. On the day of transfection, the cells were incubated with CRT-NPs (1 μg DNA per well) at 37° C. and 5% CO₂ for 48 h. Cells were then harvested and re-suspended in sterile PBS and transferred into 0.5 ml thin-walled PCR tube. The tube was placed vertically with its conical bottom aligned within the beam focus of the FUS transducer for 15 min as described previously [26, 35]. Temperature elevation (^{˜}42-45° C.) in the cell suspension with FUS was monitored using a fiber optic temperature sensor. Following FUS, the treated cells were incubated for an additional 24 h at 37° C. and 5% CO₂. Non-transfected cells were used for control. Finally, the surface expression of CRT and CD47 (n=3) was determined using flow cytometry. Stained cells were run in an LSRII analyzer (BD Biosciences, Franklin Lakes, N.J.), and datasets were analyzed using FlowJo software v.10.2 (Treestar Inc, Ashland, Oreg.). Compensations were performed with single-stained UltraComp eBeads or cells. For all channels, positive and negative cells were gated on the basis of fluorescence minus one control. In addition, B16F10 cells transfected with CRT-NPs (followed by ±FUS) were gently rinsed, scraped, and re-suspended in sterile PBS. 4×10⁶ CRT-NPs and CFUS treated B16F10 cells (^{˜}50% viable assessed by trypan blue) were inoculated s.c. in the flank region of each mouse as a vaccine (n=5 mice/group). Tumor growth was monitored for 4 weeks. After 4 weeks, mice were sacrificed.

Mouse Melanoma CRT-NP and FUS Administration, and Assessment Methodology in Primary and Re-Challenge Therapeutic Studies

All animal-related procedures were approved and carried out under the guidelines of the Oklahoma State University Animal Care and Use Committee. B16F10 cells in DMEM supplemented with 10% v/v fetal bovine serum (FBS) and 1% v/v streptomycin/penicillin at 80-90% confluency were harvested, washed, and diluted with sterile cold PBS to generate a dose of 0.5×10⁶ cells in 50 μL per mouse. Tumor cells were injected in the flank region using a 27-gauge needle (BD, Franklin Lakes, N.J.). Mice tumor volume was measured daily by serial caliper measurements (General Tools Fraction™, New York, N.Y.) using the formula (length×width²)/2, where length was the largest dimension and width was the smallest dimension perpendicular to the length. Treatments were initiated when tumors reached a volume of 40-60 mm³ (n=4-7). The following groups were compared: 1) Control, 2) FUS, 3) CRT-NP, and 4) CFUS. Three CRT-NP intratumoral injections (20 μg DNA per injection) were given 2 days apart. FUS hyperthermia (^{˜}42-45° C.) was applied 24 h after each CRT-NP injection. FUS or CRT-NPs alone cohorts received three treatments on alternating days. Untreated tumor-bearing mice served as controls for evaluation of immune changes and abscopal effects. For tumor re-challenge, tumor bearing mice (n=4-5) were injected with 1×10⁵ cells/50 μL s.c. on the contralateral flank 7 days post-treatment of primary tumors as previously reported. The differences between groups in resistance to rechallenge at distant site were assessed.

Evaluation of ICD Mediated Immune Effects in Treated Tumors, Lymph Node and Spleen Tissues Using Flow Cytometry

For in vivo studies, mice were sacrificed 26-28 days post inoculation, and the tumors (primary), tumor draining lymph nodes (dLNs), and the spleen were excised, weighed, and processed for flow cytometry, western blot, and immunofluorescence. For flow cytometry, harvested tissues were used on the same day. Single-cell suspensions obtained from mechanical disruption of the tumors followed by enzymatic digestion (200 U/mL collagenase IV; Life Technologies, NY) were filtered through a 70 μm cell strainer (Corning Inc, Corning, N.Y.). Cells were stained with combinations of the indicated fluorochrome-conjugated anti-mouse antibodies for 30 min in the dark on ice. Antibody combinations used to distinguish immune cell populations were as follows: CD45+(Tumor infiltrating leukocytes; TILs); CD3+, CD4+(CD4+ T or helper Th cells); CD3+, CD8+(CD8+ T cells); CD11b+, F4/80+(macrophages); CD11b+, F4/80+, MHCIIhi (M1 macrophages) and CD86+(activated M1 macrophages); CD11b+, F4/80+MHCII lo/neg, CD206+(M2 macrophages); CD11b+ CD11c+, F4/80−, MHCII+(dendritic cells); CD11b+Gr-1+(Myeloid-derived suppressor cells, MDSCs); PD-L1+ TILs and tumor cells; and PD-1+ CD3+CD8+ T cells. For detecting IFN-γ, Granzyme-B, and Foxp3 positive T cells, cells were washed after surface marker staining, fixed and permeabilized with transcription factor buffer set (BD Biosciences, Franklin Lakes, N.J.), and incubated with APC Cy7 anti-IFN-γ, FITC anti-Granzyme-B, or PE anti-Foxp3 antibody for 30 min in the dark on ice. Stained cells were run in an LSRII analyzer (BD Biosciences, Franklin Lakes, N.J.) within 24 h. Compensations were performed with single-stained UltraComp eBeads or cells. Datasets were analyzed using FlowJo software v.10.2 (Treestar Inc, Ashland, Oreg.). For all channels, positive and negative cells were gated on the basis of fluorescence minus one control.

Establishment of Melanoma Specific Immunity of CRT-NP and CFUS Treatments

To determine melanoma specific immunity, spleen (n=3-4) and dLNs (n=3) from the surviving mice were stimulated ex-vivo with melanoma specific differentiation antigen tyrosinase-related protein 2 (TRP-2) peptide for 8 h to evaluate generation of TRP-2 melanoma antigen specific immunity. Briefly, 1-2×10⁶ splenocytes and dLN cells were incubated with 5 μg/ml TRP-2 peptide for 8 h in the presence of Brefeldin A (eBioscience, 1000× solution) at 37° C. and 5% CO₂. Treated cells were washed with PBS and stained with CD45, CD3, CD4, CD8, and IFN-γ antibodies for flow cytometry. For immunofluorescence and western blot estimation, tissues were snap-frozen in liquid nitrogen and stored at −80° C. until further analysis.

Assessment of PD-L1 Expression in Tumor Lysates by Western Blotting

For the assessment of PD-L1 expression (n=4-7), crude membranes were extracted using Mem-PER™ Plus Membrane Protein Extraction Kit (ThermoFisher Scientific) according to the manufacturer's instructions. Equal amounts of protein (10 μg) were resolved in 4-20% Mini-PROTEAN polyacrylamide gel (BioRad, CA) and were subsequently transferred onto a nitrocellulose membrane using a BioRad Turbo Trans system. After blocking (5% non-fat dry milk), membranes were incubated at 4° C. overnight with anti-mouse PD-L1 (1:1000, Sino Biological, 50010-732). anti-mouse GAPDH (1:10000, lnvitrogen, AM4300) and anti-Na+/K+ATPase (1:3000, Abcam, ab7671 and 1:1000, Cell Signalling Technology, 3010). This was followed by incubation with secondary antibodies conjugated to horseradish peroxidase (1:10000, rabbit or goat anti-mouse, Jackson ImmunoResearch Inc., PA) at room temperature for 1 h. The blots were developed using the ECL kit (Thermo Scientific, Rockford, Ill.) and imaged by Amersham imager 600 system (GE Healthcare Bio-Sciences, Uppsala, Sweden). Na+/K+ ATPase was used as a loading control. Densitometric analyses were performed using ImageJ 1.51 software (NIH), and the data were normalized relative to appropriate GADPH controls.

Immunofluorescence Staining of Tumor Sections for CRT Expression Assessment

Tumor sections of 5 μm thickness embedded in OCT were permeabilized with acetone for 5 min and incubated with 1% BSA in phosphate-buffered saline for 2 h to block non-specific protein-protein interactions. Tissue sections were incubated overnight at 4° C. with primary anti-rabbit anti-calreticulin antibody (Pierce, PA5-25922) according to the manufacturer's recommendations. This was followed by incubation with secondary antibody conjugated to Alexa Fluor Plus 647 (Thermo Scientific, A32733) at room temperature for 1 h. Fluorescently-labeled tissues were mounted with medium containing DAPI for cell nuclei visualization (Vector Laboratories). Cell nuclei were visualized at an exposure time of 5 ms (ex/em of 365/440), and CRT was imaged at an exposure 10 ms (ex/em of 650/672). Image acquisition and display parameters were constant for different groups to allow for qualitative comparison.

Analysis of 111-8 and TNF-α in Tumor Samples by ELISA

50 μl of the tumor supernatant from homogenized tumors samples were utilized for IL1-β (n=3) and TNF-α (n=7-8) using Quantikine ELISA kit (R&D Inc., MN) according to the manufacturer's instructions.

Statistical Analyses

Statistical analyses were performed using GraphPad Prism 8.0 software (GraphPad Software Inc, La Jolla, Calif.). Data are presented as mean±SEM unless otherwise indicated. For analysis of 3 or more groups, a one-way ANOVA test was performed followed by Fisher's LSD without multiple comparisons correction. The overall P value for Kaplan-Meier analysis was calculated by the log-rank test. Analysis of differences between 2 normally distributed test groups was performed using an unpaired t-test assuming unequal variance. Correlations between PD-1+CD8+ T cells and granzyme B+ CD8+ T cells were analyzed using a Pearson correlation test, pooling data across the different treatment groups. P values less than 0.05 were considered significant.

Results

CRT-NPs Efficiently Encapsulated the Plasmid and Induced Intracellular CRT Expression, and Synergized with FUS In Vitro and In Vivo by Modulating CD47 to CRT Ratio

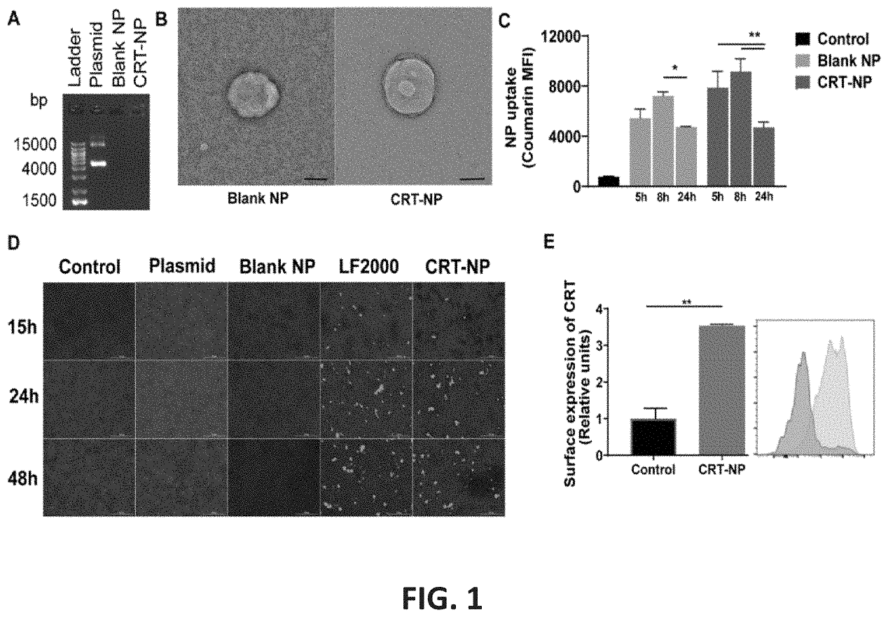

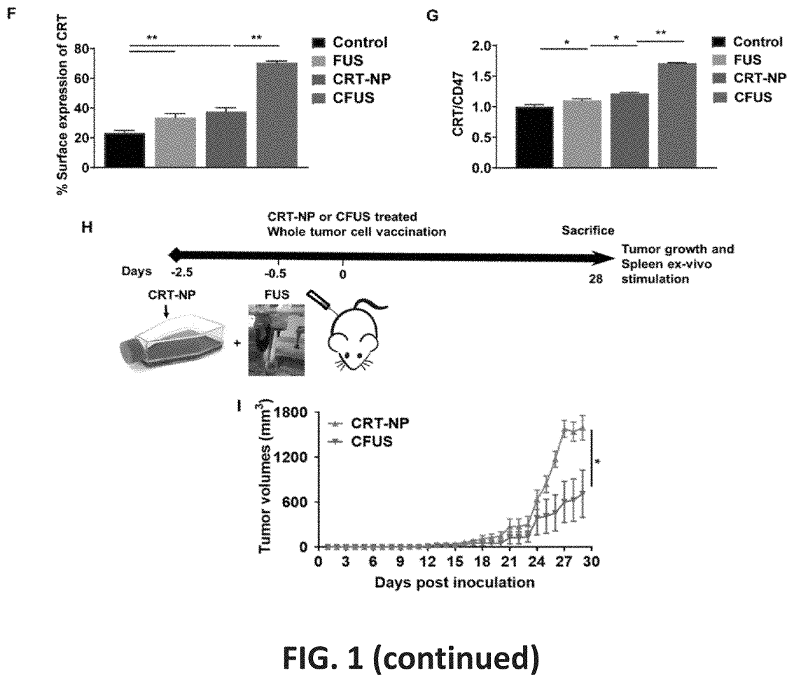

For CRT-NP synthesis, CRT plasmids were encapsulated in the cationic liposomes composed of DOTAP and cholesterol (10:1; lipid: plasmid; wt.: wt). Compared to the free CRT plasmid, agarose gel electrophoresis showed that DNA migration was absent for the CRT-NPs (Panel (A) of FIG. 1), indicating efficient plasmid encapsulation. The encapsulation was also evident in TEM where the CRT-NPs demonstrated a typical spherical core-shell morphology encapsulating the plasmid with an average size of ^{˜}230 nm (Panel (B) of FIG. 1). Additional characterizations by DLS in physiological buffer showed a hydrodynamic diameter of ^{˜}250 nm, zeta-potential of ^{˜}+14 my, and a PDI<0.3 for the CRT-NP, and excellent stability in physiological buffers up to several days (Table 1).

TABLE 1
Characterization of CRT-NP Stability over >2 Weeks (n = 5/day)

| | Hydrodynamic | Zeta potential | Polydispersity |
|---|---|---|---|
| Day | diameter nm | mV | index |
| 0 | 247.8 ± 3.1 | 12.7 ± 26 | 0.2 ± 0.01 |
| 2 | 282.4 ± 2.2 | 10.8 ± 1.2 | 0.2 ± 0.03 |
| 4 | 262.5 ± 2.7 | 13.5 ± 2.4 | 0.1 ± 0.01 |
| 5 | 266.6 ± 3.7 | 22.3 ± 2.0 | 0.2 ± 0.01 |
| 6 | 248.0 ± 2.6 | 18.6 ± 3.4 | 0.2 ± 0.02 |
| 8 | 255.8 ± 1.7 | 13.9 ± 3.9 | 0.1 ± 0.02 |
| 10 | 252.1 ± 1.9 | 14.1 ± 0.9 | 0.2 ± 0.02 |
| 12 | 245.5 ± 1.4 | 9.2 ± 1.0 | 0.2 ± 0.02 |
| 16 | 259.9 ± 1.7 | 16.3 ± 1.8 | 0.1 ± 0.02 |

NPs with a positive charge are efficiently taken up by cells. To determine whether this was true in case of CRT-NPs, fluorescence imaging and flow cytometry analysis of B16F10 melanoma cells incubated with CRT-NPs were performed. A significantly enhanced uptake of coumarin-labeled CRT-NPs at 5 h relative to untreated control was noted with flow cytometry. Also, the MFI signals plateaued at ^{˜}8 h, and started to decrease at 24 h, indicating NP lysis (Panel (C) of FIG. 1). To assess whether the enhanced NP uptake translated into an increased CRT expression in the B16F10 cells relative to the un-transfected control, fluorescence imaging of the treated cells was performed at 15, 24, and 48 h post transfection. Compared to control, plasmid, and blank NP, the data indicated a significant and progressive increase in CRT expressions over 48 h similar to the LF2000 positive control (Panel (D) of FIG. 1). These were also verified in quantitative flow assays where the CRT expression was found to be ^{˜}2-fold higher for the CRT-NPs compared to un-transfected control (Panel (E) of FIG. 1). Next, the role of FUS in CRT-NP therapy was assessed. Adding FUS to CRT-NPs (CFUS) can hypothetically enhance membrane translocation of CRT and modulate the CRT/CD47 axis by thermal effect. To assess this mechanism, B16F10 cells transfected with CRT-NPs were exposed to FUS heating in vitro (42-45° C.). Data indicated that CRT-NP+FUS (CFUS) was most effective in inducing CRT expressions (3-fold higher) compared to the untreated control (Panel (F) of FIG. 1). Also, in contrast to the CRT-NPs for which the enhanced expression of CRT was accompanied by a concurrent upregulation of CD47, the CFUS treatment increased the CRT without significantly changing the CD47 membrane expression, thereby resulting in a 1.5-fold increase in CRT/CD47 ratios compared to the control, FUS and CRT-NP (Panel (G) of FIG. 1). Finally, to confirm whether downregulating the CD47 expression induced tumor regressions in vivo, the mice (n=5) were inoculated subcutaneously with CRT-NP and CFUS treated cells in the flank regions (Panel (H) of FIG. 1). A significantly superior tumor regression for CFUS relative to CRT-NP treated cells (n=5) was noted in the mice over 4-week, indicating that CFUS directly prevented the CD47 counteraction of CRT expression in melanoma cells to improve the therapeutic response (Panel (I) of FIG. 1).

Local CRT-NP and CFUS Tumor Therapy Enhanced ICD and Therapeutic Efficacy In Vivo

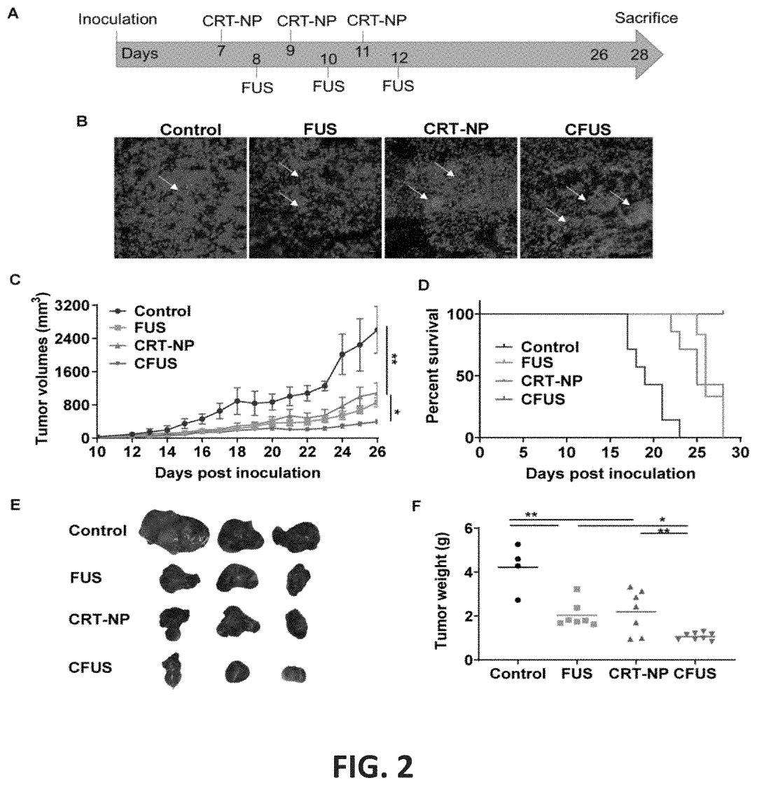

CRT-expression and efficacy of CRT-NP and FUS heating was evaluated by tumor growth and weight measurements in B16F10 melanoma model over 26-28 days (Panel (A) of FIG. 2). When FUS was combined with CRT-NP, the CRT expression in tumors was significantly upregulated in the fluorescence imaging (Panel (B) of FIG. 2). Therapeutically, the untreated control mice exhibited pronounced increases in tumor volume (Panel (C) of FIG. 2). CRT-NP alone and FUS treatments induced significant tumor volume reductions (^{˜}50%) compared to the control but did not differ significantly from each other. In contrast, the CFUS treatments caused significant suppression of tumor growth rates (>85%) versus that seen in untreated controls and an effect that was ^{˜}50% greater than that seen with CRT-NP or FUS alone over the period of treatment. Evaluation of survival rates indicated that the CFUS treated mice (n=7) demonstrated 100% survival vs 70% for CRT-NP (2/7). In contrast, FUS (0/7) and control (0/7) were ineffective in tumor control and survival compared to CRT-NP and CFUS (Panel (D) of FIG. 2). Furthermore, CFUS significantly decreased the tumor weight to a greater extent by visual and statistical measures compared to all other groups (Panels (E)-(F) of FIG. 2).

CRT-NP and CFUS Induced ICD Increased the Infiltration of Tumor Suppressing Immune Cells

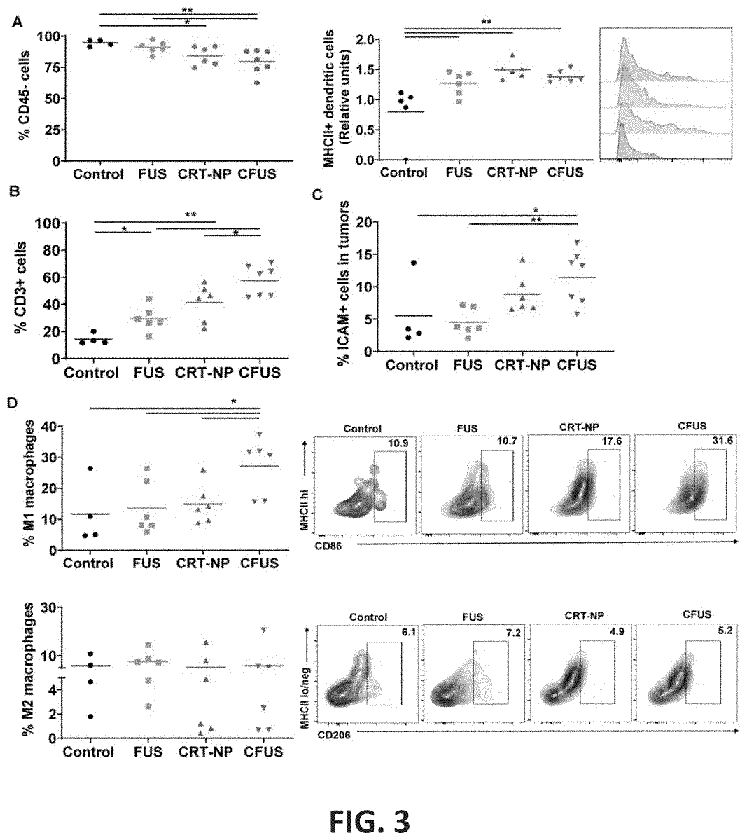

The percentage of the non-immune (tumor and fibroblast cells) and dendritic cells (DC), tumor associated macrophage (TAM) phenotypes, and T cells were determined within B16F10 melanoma tumor. The percentage of CD45 minus cells (tumor cells and fibroblasts; mean±SEM) in tumors were 95±1.32, 90.9±1.91, 84.2±3.1, and 79.5±3.6 for control, FUS, CRT-NP and CFUS, respectively. Also, the MHCII expression on DC (CD11b+ CD11c+F4/80-) was relatively higher in the treated tumors compared to untreated control, indicating activation of immune system (Panel (A) of FIG. 3). The decrease in tumor cell population for CFUS and CRT-NP strongly correlated with the infiltration of CD3+ T cells (2-3 fold, Panel (B) of FIG. 3), and overall ICAM-1 levels in tumors (2-fold, Panel (C) of FIG. 3). In addition, CFUS significantly enhanced TAMs (CD11b+F4/80+) of the M1 phenotype (MHCIIhigh; 3-fold) without impacting the M2 population (MHCIIlow/negative CD206+; Panel (D) of FIG. 3).

CRT Based ICD Improved the Local and Systemic Anti-Tumor Immunity Compared to Untreated Control

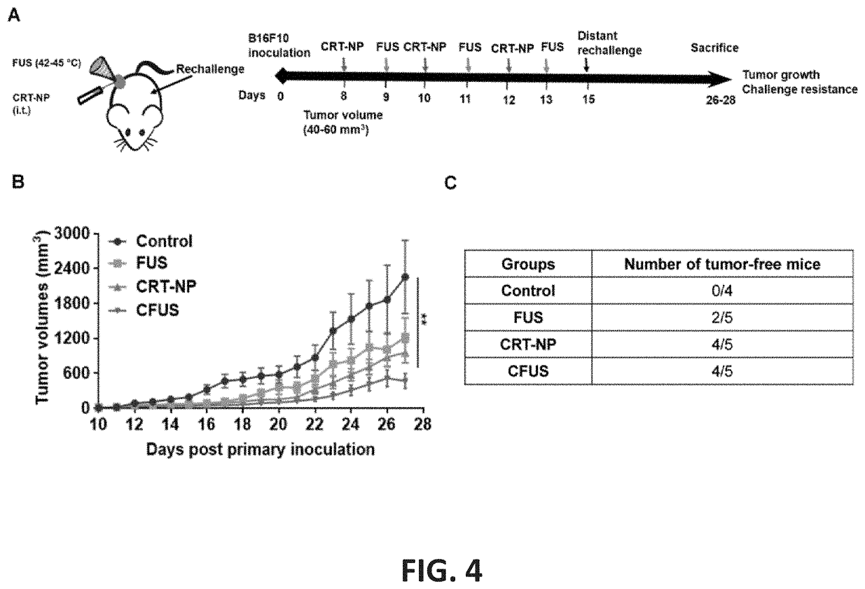

To determine the role of CRT induced ICD in inducing resistance against re-challenge, mice (n=4-5) were randomized as follows: control, FUS, CRT-NPs, and CFUS (Panel (A) of FIG. 4). Briefly, the primary tumors were treated at a volume of 40-60 mm³, and then mice were re-challenged with 1×10⁵ B16F10 cells s.c. on the contralateral flank on day 7 and observed for 14 days for tumor growth. Data indicated that the sustained pressure on the immune system by tumor re-challenge did not impact therapeutic effects at the primary treated site. Overall, CFUS was most effective in local control relative to untreated control, FUS, and CRT-NP during the longitudinal monitoring period (Panel (B) of FIG. 4). In addition, 80% of tumor-bearing mice (4/5) in CFUS and CRT-NP remained tumor-free and resisted challenge at distal site compared to 40% mice (2/5) in FUS and 0% (0/4) in the untreated control (Panel (C) of FIG. 4).

ICD Mediated by CRT-NP and CFUS Induce Melanoma Specific Immunity

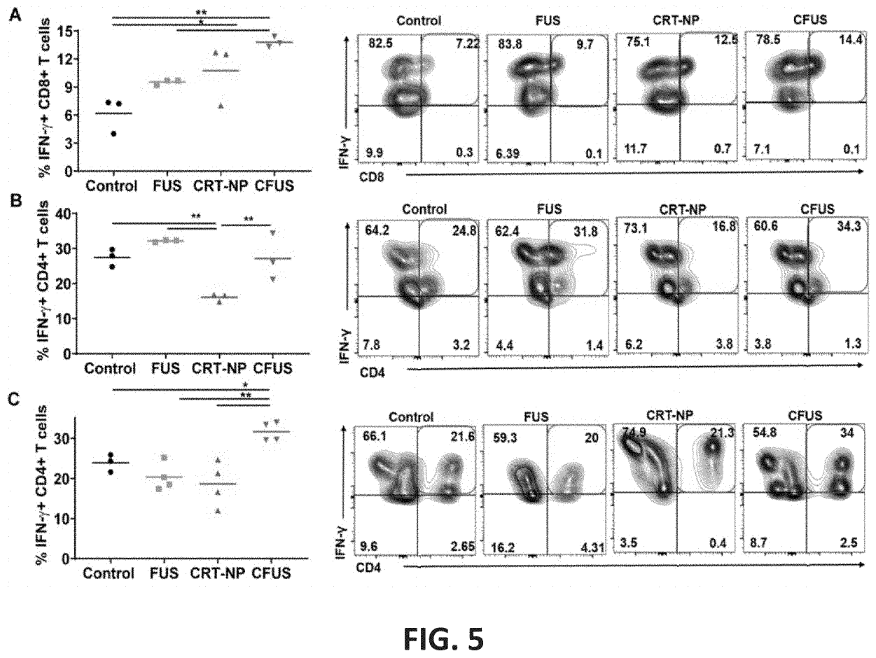

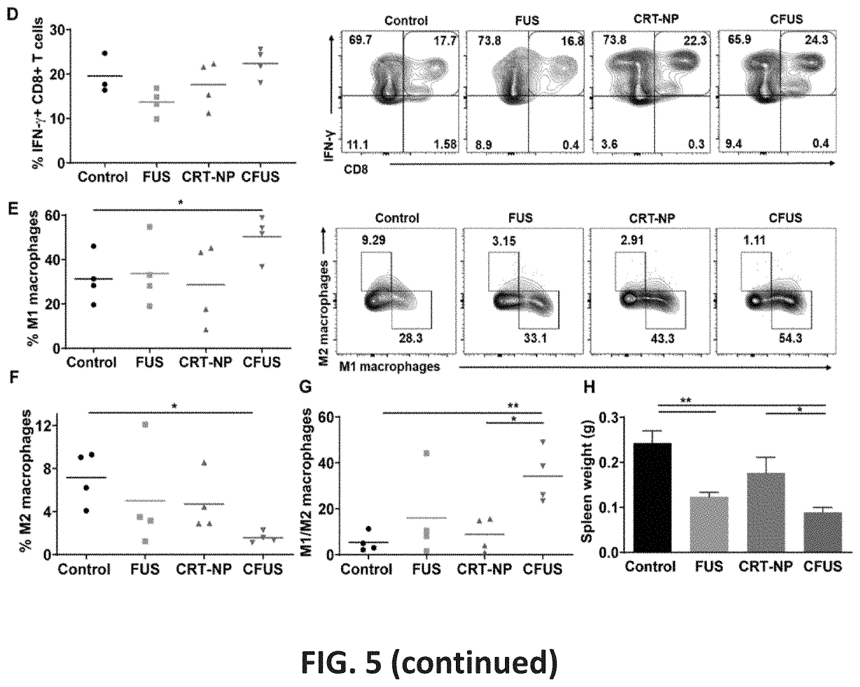

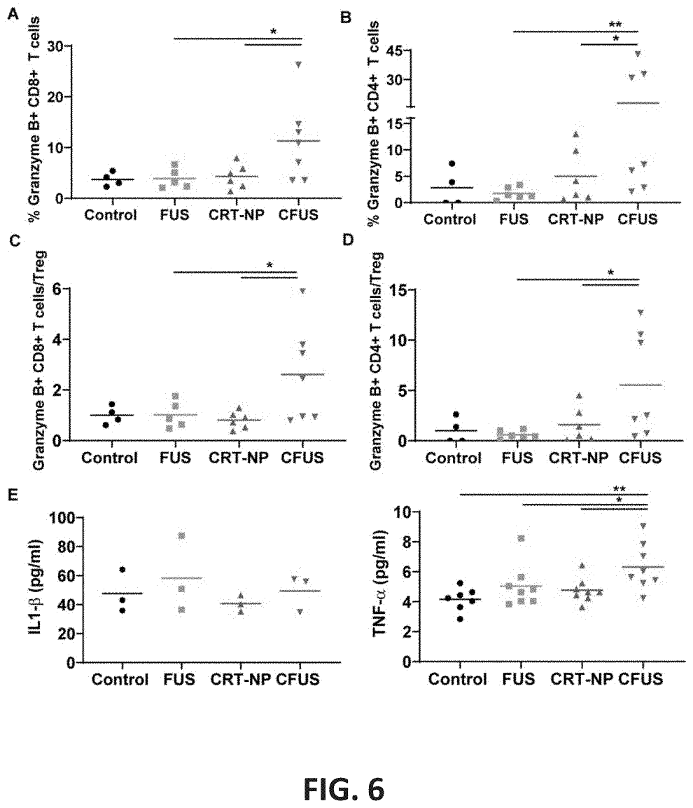

To determine melanoma antigen-specific immune response in the mice model, the production of IFN-γ from T cells in the dLNs and splenic tissue of surviving mice were assessed. An increase in IFN-γ+ CD8+ T cells (^{˜}2-fold fold) in dLNs in CRT-NP, CFUS, and FUS were observed, but not IFN-γ+ CD4+ T cells indicating the proliferation of melanoma specific cytotoxic immune cells (Panels (A)-(B) of FIG. 5). In contrast, the splenocytes from various groups that were cultured ex vivo and stimulated with TRP-2 melanocyte antigen showed a significant enhancement of IFN-γ+ CD4+ T cells for CFUS compared to CRT-NP. However, the IFN-γ+ CD8+ T cells were not altered significantly between groups (Panels (C)-(D) of FIG. 5). The splenic macrophages were also analyzed for the M1/M2 phenotype and tumor T cell phenotypes to understand why CFUS induced the most prominent antitumor effects following local treatments compared to all other groups. Results showed a 1.5-2-fold increase in M1 subsets (Panel (E) of FIG. 5) and 4-5-fold increase in M1/M2 ratio for CFUS (34.2±5.1) compared to control (5.5±2.4), FUS (16.1±8.3), and CRT-NP (8.9±3.2; Panels (F)-(G) of FIG. 5). These changes in the M1/M2 ratio accompanied a significant decrease in the splenic weight for treatment cohort's vs control (Panel (H) of FIG. 5). Importantly, for the CFUS group, the population of functional T cells demonstrated the highest population of CD3+ CD8+ and CD4+ T cells expressing granzyme B, a key activation marker involved in tumor cell lysis (Panels (A)-(B) of FIG. 6; % Granzyme B+ CD8+ T cells; CFUS: 11.29±3.0, Control: 3.72±0.68, FUS: 3.88±3.40, and CRT-NPs: 4.31±0.97 and for % Granzyme B+ CD4+ T cells; CFUS: 17.85±6.46, Control: 2.82±1.78, FUS: 1.77±0.46, and CRT-NPs 5.01±2.12). This increase in the activated T cells correlated with increased ratios of CD8+ and CD4+ T cells to Tregs for CFUS compared to other groups (Panels (C)-(D) of FIG. 6). Lastly, the immuno-activated profile in tumors accompanied an increase in the expressions of TNF-α in CFUS tumors. In contrast, the expression of IL-1β was not altered between groups (Panel (E) of FIG. 6).

Adaptive Resistance can Emerge in Melanoma Tumors Following Withdrawal of CRT-ICD Therapy.

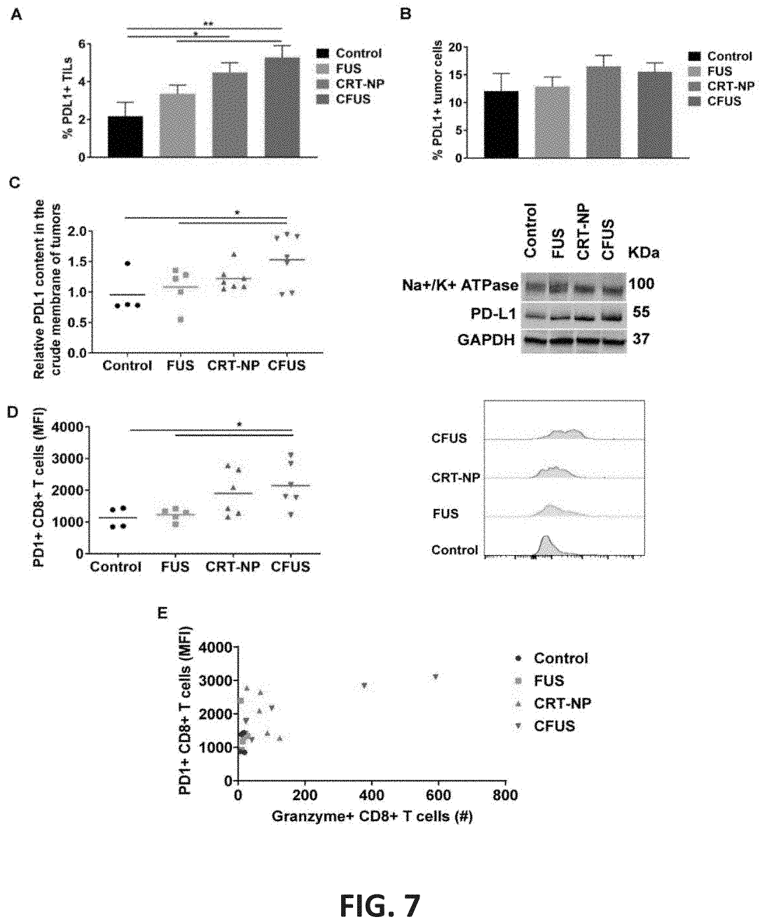

To understand the role of ICD therapy in PD-L1/PD-1 pathway on tumor cells and TILs, we assessed expression of PD-L1 and PD-1 in tumors. Flow cytometry and western blot data indicated that the PD-L1 expressions on tumor cells were not modified by the treatments, but the frequency of PD-L1+ TILs from CFUS tumors was significantly increased (^{˜}2.5 folds) compared to control (Panels (A)-(C) of FIG. 7). Additionally, CFUS treatment enhanced the median fluorescence intensity (MFI) of PD-1 on CD3+ CD8+ T cells compared to CRT-NP, control and FUS (Panel (D) of FIG. 7). This enhancement was particularly associated with the Granzyme B+ CD8+ T cells (r=0.644, p<0.01, Panel (E) of FIG. 7). Notably, the mice that expressed highest levels of Granzyme B+ and PD-1 CD8+ T cells demonstrated superior tumor regression (especially in CFUS group). In contrast, low levels of granzyme B and PD-1 expression resulted in sub-optimal therapeutic outcomes.

Discussion

The goal of this Example was to understand how CRT based ICD impacts the infiltration of immune cell, antigen cross-presentation, and antitumor immunity in murine melanoma. CRT is a pro-phagocytic signaling protein that translocates to tumor cell membranes during cellular stress to promote immunogenicity of tumors. CRT expression is mainly mediated by chemo-, radio-, and ablative therapy, but the extent of expression rates, antigen recognition, and immune cell trafficking can be highly variable. To overcome this barrier and achieve robust ICD, liposome-based CRT-NPs were developed. In vitro data indicated that the synthesized NPs successfully encapsulated the plasmid (Panels (A)-(B) of FIG. 1) and achieved high uptake and transfection efficiencies and surface exposure of CRT in melanoma cells in vitro and in vivo (Panels (C0-(E) of FIG. 1 and Panel (B) of FIG. 2). In contrast to CRT, CD47 (integrin-associated protein, IAP) acts as a negative checkpoint (“don't eat me”) of the innate immune system by interacting with signal-regulatory protein α (SIRPα) on macrophages, preventing their phagocytosis. Because CD47 counterbalances the CRT pro-phagocytic and adaptive immunity, it may impact maximum synergy and durable responses from CFUS therapies. The in vitro and in vivo studies of the present disclosure indicate that FUS directly prevented the CD47 counteraction of CRT expression in melanoma cells, significantly improving therapeutic regression of tumors (Panels (F)-(H) of FIG. 1). This Example also includes the addition of anti-CD47 antibody in the treatment regimen to shed more insights on this important phenomenon to optimize clinical outcomes, especially in scenarios where a proportional increase in the “don't-eat-me” signals, such as CD47 with CRT, are noted.

An enhanced surface translocation of CRT followed by ICD is known to activate innate and adaptive immune cells. To test whether this was true in the model system of this Example, the infiltration of M1 macrophages in the treated tumor and spleen was determined upon in-situ vaccination with CRT-NP. CRT-NP and the addition of FUS in the CRT-NP regimen achieved a 2-fold increase in M1/M2 ratio compared to control and CRT-NP treatments (Panel (D) of FIG. 3 and Panels (E)-(G) of FIG. 5), verifying prior published findings wherein an increased M1/M2 ratio was associated with improved patient survival. This phenomenon is typically attributed to the ability of M1 macrophages to secrete complement factors that facilitate phagocytosis, present antigens to T cells, and effectively shape an adaptive immune response. In contrast, high populations of M2-macrophages can promote tumor initiation, progression, and metastasis. Recent works in murine mammary, colon, and melanoma cancers have also shown the presence of non-M1/M2 macrophage subtypes rich in IFN-γ section, T-cell receptor, and CD169 expressions, and receptors with collagenous structure (MARCO) with M2-like profile. While the data in this Example indicate that M1 phenotype was enhanced, more detailed studies may be required to correctly delineate the macrophage sub-populations that are involved in antitumoral effects with ICD. Like macrophages, dendritic cells play an important role in initiating an adaptive immune response by processing tumor antigens and presenting peptide fragments to activate naive CD4+ and CD8+ T cells, aiding in the clonal expansion of cytotoxic T lymphocytic cells, and improved therapeutic outcomes. The present Example found that the treated mice tumors demonstrated a higher frequency of T cell and DCs following ICD with CRT-NP and CFUS (Panels (A)-(B) of FIG. 3). Surprisingly, no significant alterations were observed in intratumoral IL-1β levels, a pro-inflammatory cytokine produced from activated macrophages that is involved in T cell activation. This said, our phenotypic characterization of CD4+ and CD8+ T cells in tumors from the treated mice revealed up-regulation of granzyme B along with an intratumoral increase in TNF-α especially for the CFUS group compared to monotherapies (Panels (A)-(E) of FIG. 6), and this correlated strongly with the antitumor effects (FIGS. 2 and 4). IFN-γ, TNF-α, and granzyme B are typically associated with antitumor activity of cytotoxic CD8+ T cells via induction of enhanced tumor cell arrest and apoptosis. Together, the data of the present Example indicate that ICD induces tumor inflammation via multiple interrelated pathways, leading to tumor regression. In addition, FUS acoustic parameters may be adjusted to further affect the CRT translocation rates, cytokine expressions, and immune activation and to delineate the role of FUS parameters on immune infiltrations and their relationships with CD47, granzyme B, and cytokine expression.

A key challenge in immunotherapy regimens is the generation of tumor-specific T cells against distant untreated tumors. It was found that transfection of B16F10 cells with CRT-NP and combination with FUS heating significantly enhanced the populations of IFN-γ+ CD4+ and CD8+ T cells (^{˜}1.5-2) in dLN and splenic tissues. IFN-γ producing T-cells promote the priming and expansion of cytotoxic cells. While not wishing to be bound by a particular theory, it is proposed that CRT-ICD delays tumor growth in distant untreated site by increasing the tumor antigen-specific T-cell quantity and quality (Panels (A)-(D) of FIG. 5). Finally, the role of immune checkpoints such as PD-L1/PD-1 that negatively influences innate and adaptive immune system was also assessed. When antigen-specific T cells surround the tumor cells, the tumor cells along the T-cell rich margin upregulate PD-L1 as an immune evasive mechanism. This compensatory elevation in PD-L1 expression is thought to be due to presence of activated T-cells and chronic IFN-signaling, leading to impaired tumor cell killing. It was observed that the treatments of the present disclosure upregulated PD-1/PD-L1 protein on tumor infiltrating lymphocytes (TILs) compared to untreated control (Panels (A)-(B) of FIG. 7). Additionally, the CD8+ T cells showed increased granzyme B and IFN-γ expression upon CFUS therapy (Panel (C) of FIG. 6). Thus, it was hypothesized that the ICD induced T cell activation and the concurrent presence of chronic IFN-γ secretion can contribute to PD-L1 expression and development of an adaptive immune resistance mechanism, and this may influence the overall therapeutic outcomes. To overcome this barrier, the inclusion of checkpoint inhibitors in the ICD regimens can likely result in significantly improved outcomes in such cases. This is supported by the data in the present Example, where the tumors that showed superior regression with ICD (especially CFUS) contained higher populations of PD-1+/PD-L1+ CD8+ T cells and activated CD8+ T cells (granzyme expressing; Panel (D) of FIG. 7). Unlike tumor cells, PD-L1 expression on TILs has been associated with favorable prognosis in head and neck cancer and melanoma. Likewise, high PD-L1+ expressing metastatic melanoma achieved a superior clinical response to check point blockades compared to PD-L1− metastatic melanoma. Thus, it is indicated that the presence of activated T-cells and PD-L1+ TIL cells and inclusion of checkpoint blockade can mitigate adaptive resistance effects to some extent.

In summary, the in vitro and in vivo data of this Example indicate that CRT-based ICD promotes antigen presentation and infiltration of activated CD8+ T cells in tumors. Adding FUS to CRT-NP therapy modulates the CRT-CD47-PD-L1 axis, improving the overall local and systemic therapeutic effects in melanoma. ICD synergism with checkpoint blockades, anti-CD40 antibodies, and different FUS parameters can provide more mechanisms to mitigate adaptive immune resistance, maximizing therapeutic effect and survival.

### Example 2

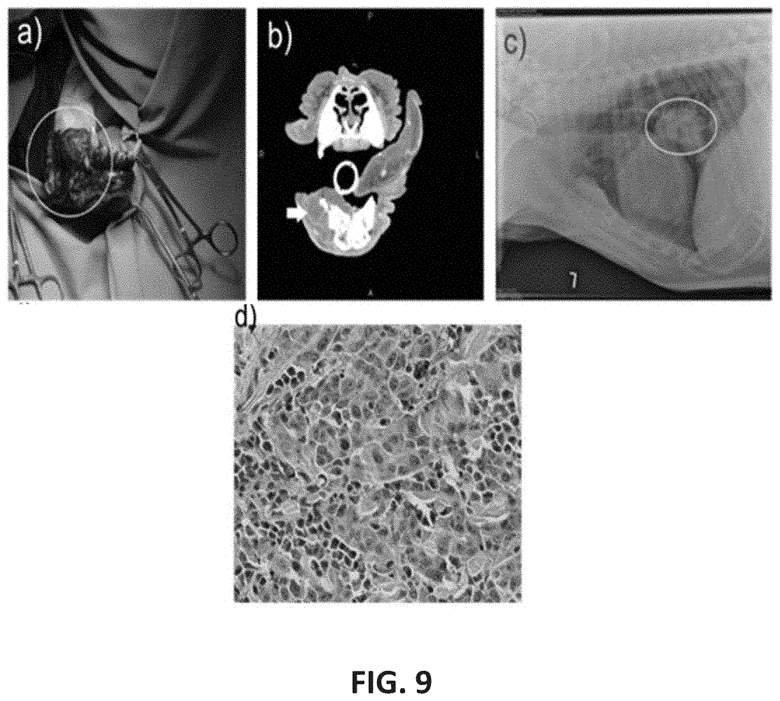

Dogs are genetically variable animals with annual cancer incidence of 4.2 million dogs/year (^{˜}5,300/100,000 population rate), and tumor type and site situations comparable to those in human cancer (1.66 million humans; approx. 500/100,000 population rate) in the USA. Canine oral melanoma is very similar in behavior to aggressive human dermal melanoma originating in skin (FIG. 9).

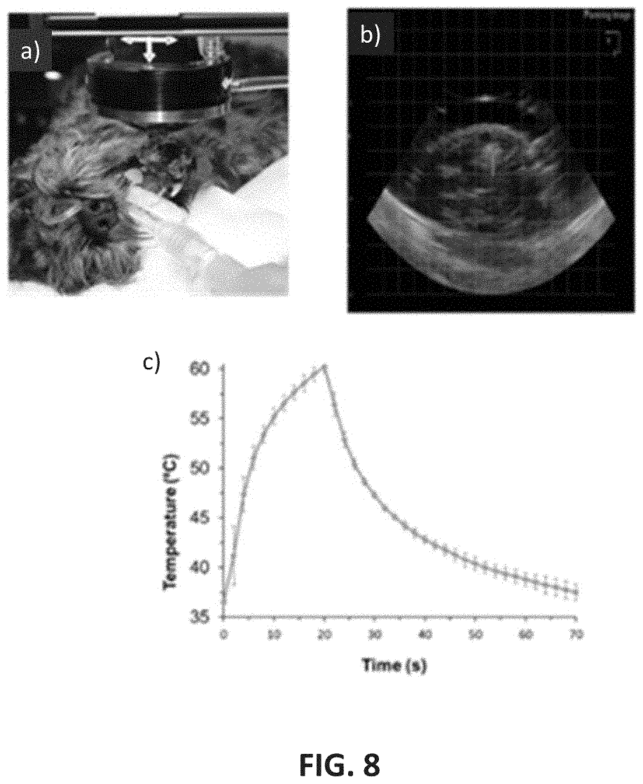

For dog oral melanoma treatments, a dry type platform with 1 Mz transducer capable of handling animals up to 501b was used (FIG. 8). The sophisticated software-controlled system is used to optimize tumor damage and spare normal tissue. CT/MRI imaging has been used for treatment planning and follow-up.

Following sedation/anesthesia, oral melanoma regions of dogs were cleaned with standard surgical disinfectant and saline. Degassed gel was used to provide acoustic coupling, and sonications were targeted to the center of oral melanoma tumor using VIFU planning software under ultrasound guidance to define target boundary and slice distance in X, Y, and Z directions for automatic rastering of the transducer, as shown in an intubated dog lying on the right side (Panel (a) of FIG. 8). Panel (b) of FIG. 8 demonstrates an ultrasound image of the oral melanoma tumor. This application can be used in real-time to monitor the ongoing FUS treatment. A 3×3-raster pattern of focal points along the x axis was used to generate ablative temperatures in each focal region in x, y, and z (Panel (b) of FIG. 8). Tumor temperatures were monitored using fiber optic probes placed into the tumor. FUS treatment parameters used were as follows: 5 Hz pulse repetition frequency (PRF), 50% duty cycle, and 90 W acoustic power to achieve a mean target temperature of 55-60° C. at the focus for 15-20 seconds (Panel (c) of FIG. 8).

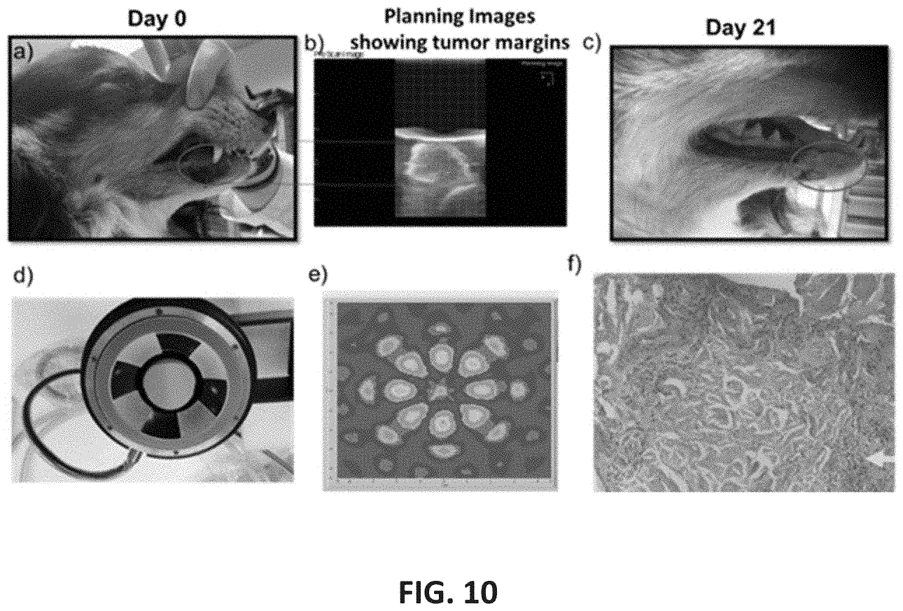

In this example, canine tumor treatment was performed on a 9-year-old Shetland Sheepdog in the OSU Teaching Hospital. The castrated male dog presented with a benign plasmacytoma mass (Length=25 mm. Width=20 mm) on the lower right lip margin at the level of the canine/premolars that extended into the gum margin. In contrast to surgery that would have needed a large region of tissue to be resected, the owner was offered a non-surgical and non-ionising FUS ablation treatment option. For treatments, the Alpinion FUS transducer of 1 MHz central frequency fitted with a sector vortexed lens that allows 5×5×10 mm focal spot treatments (FIG. 10, 5 Hz frequency, 50% duty cycle, 90 W acoustic power, and a peak positive/negative pressure of 6.15/−4.41 MPa) was used. For FUS exposure, the tumor was aligned at a fixed focal depth for efficient coverage, and temperature in the treated region was measured using a fiber-optic temperature sensor. 2-ablative focused ultrasound treatment (covering 50% of tumor volume) was performed for 1-3 min in the tumor core. The benign tumor responded amazingly well clinically, and the immunological reaction in the tumor and peri-tumor tissue indicated enhanced immune cell infiltration 3 weeks post-treatment.

The FUS ablation treatment approach outlined above can be extended in the context of malignant melanoma tumors, and to assess benefits of CFUS with radiological, immunological and histopathological means. All patients are recruited through OSU Teaching hospital. The trial uses privately owned dogs with metastatic oral melanoma. The primary endpoints are local and metastasis control. The pilot clinical trial in client-owned dogs with oral melanoma uses treatment parameters optimized in mouse studies and includes CFUS and CFUS/PDL-1 therapy with 6 dogs per group. For PDL-1 therapy, human anti-PDL-1 is administered, as it has been shown to cross-react with dog PDL-1 receptors. Since client-owned dogs cannot be left untreated in the clinical trial, a surgery-only group serves as a control.

CT scans are used to coregister with real-time ultrasound imaging at the time of FUS to improve ultrasound imaging and planning and monitoring of ongoing oral treatment. Vital signs are monitored using a fiber optic cuff placed around a shaved front paw. Degassed gel is used to provide acoustic coupling, and FUS sonications are targeted to the center of oral tumors using VIFU planning software. CRT-NP (intratumoral) and PDL-1 (intraperitoneal) are delivered four times over a two-week period, typically on a Monday-Friday schedule. Each 20 μgCRT-NP/cm³ tumor injection is placed in 3 intratumoral locations (approximately 35 μl/injection point). CRT-NP is delivered 24 h prior/after FUS and 2, 5, 9 days post-FUS treatment. As part of post-treatment examination all dogs receive a complete physical exam at each assessment point and a CT exam 12 weeks post-treatment.

Thus, in accordance with the present disclosure, there have been provided compositions, kits, and systems, as well as methods of producing and using same, which fully satisfy the objectives and advantages set forth hereinabove. Although the present disclosure has been described in conjunction with the specific drawings, experimentation, results, and language set forth hereinabove, it is evident that many alternatives, modifications, and variations will be apparent to those skilled in the art. Accordingly, it is intended to embrace all such alternatives, modifications, and variations that fall within the spirit and broad scope of the present disclosure.

What is claimed is:

1. A composition comprising:
a nanoparticle comprising a calreticulin gene or a fragment or variant thereof.

2. The composition of claim 1, wherein the nanoparticle comprises a liposome.

3. The composition of claim 2, wherein the liposome is a cationic liposome.

4. The composition of claim 2, wherein the liposome comprises DOTAP (1,2-dioleoyl-3-trimethylammonium-propane) and cholesterol.

5. The composition of claim 1, wherein the nanoparticle has a hydrodynamic diameter in a range of from about 200 nm to about 300 nm.

6. The composition of claim 1, wherein the calreticulin gene has the nucleotide sequence of SEQ ID NO:1.

7. The composition of claim 1, wherein the calreticulin gene encodes the amino acid sequence of SEQ ID NO:2.

8. The composition of claim 1, wherein the nanoparticle has a Zeta potential in a range of from about +1 mV to about +20 mV and a polydispersity index of less than about 0.3.

9. The composition of claim 1, wherein the composition is substantially stable for at least 14 days at room temperature.

10. The composition of claim 1, further defined as a pharmaceutical composition, and wherein the pharmaceutical composition further comprises a pharmaceutically acceptable carrier or excipient.

11. The composition of claim 10, wherein the pharmaceutical composition is formulated for injection into a patient.

12. The composition of claim 10, further comprising at least one of a PD-1 inhibitor, a PDL-1 inhibitor, or a CD47 inhibitor.

13. A method, comprising the steps of:
administering a pharmaceutical composition to at least a portion of a patient, wherein the pharmaceutical composition comprises a nanoparticle comprising a calreticulin gene or a fragment or variant thereof.

14. A method, comprising the steps of:
(i) administering a pharmaceutical composition to at least a portion of a patient, wherein the pharmaceutical composition comprises a nanoparticle comprising a calreticulin gene or a fragment or variant thereof; and
(ii) administering focused ultrasound to the patient so as to heat at least a portion of a tumor and/or surrounding tissue; and
wherein steps (i) and (ii) are performed simultaneously or wholly or partially sequentially.

15. The method of claim 14, wherein at least a portion of the tumor is heated to a target temperature in a range of from about 40° C. to about 60° C.

16. The method of claim 14, wherein the focused ultrasound emits a pulse repetition frequency in a range of from about 1 Hz to about 100 Hz, a duty cycle in a range of from about 10% to about 100%, and an acoustic power in a range of from about 1 W to about 100 W.

17. The method of claim 14, wherein steps (i) and (ii) are performed wholly or partially sequentially, and wherein step (i) is performed prior to step (ii).

18. The method of claim 14, wherein steps (i) and (ii) are performed wholly or partially sequentially, and wherein step (ii) is performed prior to step (i).

19. The method of claim 14, further comprising the step of repeating steps (i) and (ii) one or more times.

20. The method of claim 11, further comprising the step of:
administering to the patient at least one of a PD-1 inhibitor, a PDL-1 inhibitor, or a CD47 inhibitor.

21-26. (canceled)
